(19) United States
(12) Patent Application Publication
(10) Pub. No.: US 2024/0246126 A1
(43) Pub. Date: Jul. 25, 2024

(54) PROBE DISINFECTING APPARATUS AND METHOD OF USE

(71) Applicant: CS MEDICAL, LLC, Creedmoor, NC (US)

(72) Inventors: John Martin Jans, Hillsborough, NC (US); Michael Wayne Guin, Sanford, NC (US); Andrew Justin Fiest, Raleigh, NC (US); Mark Ronald Hoffmann, Highland Park, IL (US); Charles Adam Langley, Raleigh, NC (US)

(73) Assignee: CS MEDICAL, LLC, Creemoor, NC (US)

(21) Appl. No.: 18/421,532

(22) Filed: Jan. 24, 2024

Related U.S. Application Data
(60) Provisional application No. 63/441,059, filed on Jan. 25, 2023.

(51) Int. Cl. B08B 11/02; A61L 2/04; A61L 2/18; A61L 2/26; B08B 3/10; B08B 13/00

(52) U.S. Cl. CPC B08B 11/02; A61L 2/04; A61L 2/18; A61L 2/26; B08B 3/10; B08B 13/00; A61L 2202/123; A61L 2202/14; A61L 2202/15; A61L 2202/17; A61L 2202/24

(57) ABSTRACT

An apparatus and method for disinfecting probes. A probe may be positioned using a probe positioning system that locates the probe in the proper location for disinfecting. The probe may be subjected to a cleaner and/or rinsing liquid to remove foreign materials (e.g., bioburden, soil, and other like material) from the probe before disinfecting. After rinsing, a disinfectant is applied to the probe via one or more nozzles. The disinfectant is applied to the probe for a specified amount of time to disinfect the surface of the probe, and afterwards the probe is rinsed thoroughly to remove the disinfectant from the probe. The rinsing liquid and the disinfectant liquid are held in separate fluid circuits, yet heated to their respective ideal temperatures using the same heater assembly, the heater assembly having heating element shared by both fluid circuits.

### FIELD

This application relates generally to the field of cleaning and disinfecting probes, and more particularly to apparatuses and methods for disinfecting a probe by positioning the probe using a probe positioning system, a dual heater apparatus to mix and route the flow of rinsing and disinfectant fluid, and/or nozzles for dispensing liquids for rinsing and disinfecting the probes.

### BACKGROUND

Some probes, and in particular channel-less ultrasound transducer probes that are inserted into a patient require disinfection after use with the human body. Traditional disinfectant application processes may not be effective due at least in part to properly applying the disinfectant and/or requiring lengthy preparation times to achieve the necessary temperatures required for high-level disinfection, which lead to energy inefficiency and unnecessary failure points. Accordingly, there is a need for improved probe disinfecting apparatuses and methods.

### BRIEF SUMMARY

Embodiments of the invention are directed to apparatuses and methods for disinfecting probes, by providing a pre-rinsing and/or cleaning process (in some embodiments) before submitting the probe to the disinfecting process, and/or providing a post-disinfecting rinsing process. The probe may first be positioned within the disinfecting apparatus through the use of a probe positioning system that properly locates the probe within the disinfecting apparatus to optimize the dispensing of the liquids onto the probe, such as through the use of nozzles. Moreover, the disinfecting process may be improved though use a of singular heater, and as such the liquids (e.g., disinfecting liquid and cleaner liquid and/or rinsing liquid) may share a singular heater, the heater heating liquids simultaneously while only using one heating element. In some embodiments, the heater controls the temperature of a first liquid (e.g., the disinfecting liquid, or the like), while the heater and a quenching liquid control the temperature of the second liquid (e.g., the rinsing liquid, or the like).

Before the disinfecting process begins, the probe is positioned within the disinfecting apparatus, in some embodiments through the use of a probe positioning system to control the location of the probe with respect to the components (e.g., nozzles, mister, dispenser that fill the reservoir, or the like) that provide the liquids to the probe. After the probe is inserted into the apparatus, in some embodiments, a rinsing and/or cleaning is first utilized to remove foreign material (e.g., bioburden, soil, and the like) from the probe after the probe is removed from the patient and/or after the probe is pre-cleaned using other manual (e.g., cleaning or wiping) and/or automated processes. For example, in some embodiments a fluid (e.g., heated water, or other fluid) may be deposited on the probe through the use of nozzles (e.g., using a conical, overlapping spray pattern, or the like). Additionally, or alternatively, a cleaner may be used to automatically clean the probe after rinsing and/or before disinfecting. The cleaner may be a detergent or a detergent with one or more enzymes to enhance cleaning. The multiple enzymes in the cleaner rapidly attack soils and include low foam properties for effective recirculation for various cycles of cleaning the probe. Furthermore, the probe may be rinsed after the cleaning step to remove the residual cleaner from the probe and from within the fluid circuit.

After the rinsing, cleaning, and/or post-cleaning rinsing (to the extent one or more of these are utilized), the probe undergoes a disinfection process (e.g., a high-level disinfection process, or the like). The disinfectant is deposited onto the probe, such as by multiple nozzles directed at the probe. The disinfectant may be applied in a conical, overlapping spray pattern, for a specified amount of time, in order to provide coverage over the surfaces of the probe. After the disinfectant cycle, the probe is thoroughly rinsed to remove the disinfectant from the probe and from within the fluid circuit. That is, like the disinfectant, the rinsing may be applied in a conical overlapping spray pattern, for a specified amount of time, or for specific volumes of liquid.

In one aspect, the present disclosure embraces an apparatus for disinfecting a probe. The apparatus may include a probe reservoir assembly, wherein the probe reservoir assembly is configured for securing the probe and dispensing a liquid onto the probe, wherein the liquid is a disinfectant liquid or a rinsing liquid, a disinfectant assembly operatively coupled to the probe reservoir assembly, wherein the disinfectant assembly is configured to provide the disinfectant liquid for the disinfecting of the probe, and a rinsing assembly operatively coupled to the probe reservoir assembly, wherein the rinsing assembly is configured to provide the rinsing liquid for rinsing the probe.

In some embodiments, the probe reservoir assembly may include a probe reservoir, a securing member for securing the probe within the reservoir, and one or more nozzles that dispense the liquid on the probe within the probe reservoir, wherein the liquid is the disinfectant liquid for the disinfecting or the rinsing liquid for the rinsing, and wherein the one or more nozzles dispense the liquid in a conical pattern.

In some embodiments, or in combination with any of the previous embodiments, the one or more nozzles include a plurality of nozzles, wherein the plurality of nozzles provide overlapping coverage of the liquid on the probe, and wherein the liquid runs-off the probe by gravity.

In some embodiments, or in combination with any of the previous embodiments, the apparatus may further include a heater assembly having a heater having a heating element, a first circuit operatively coupled to the disinfectant assembly, and a second circuit operatively coupled to the rinsing assembly.

In some embodiments, or in combination with any of the previous embodiments, the heater may include a first channel for the first circuit, wherein the first channel receives less than half of the heat from the heating element, and a second channel for the second circuit, wherein the second channel receives greater than half of the heat from the heating element.

In some embodiments, or in combination with any of the previous embodiments, the heater assembly may further include a first temperature sensor for the first circuit, and a disinfectant temperature of the heated disinfectant is controlled by electronically adjusting the heater element based on the first temperature sensor.

In some embodiments, or in combination with any of the previous embodiments, the heater assembly may further include a second temperature sensor for the second circuit, and wherein a rinsing temperature of the rinsing liquid is controlled by adjusting water suppled to the rinsing liquid for quenching based on the second temperature sensor.

In some embodiments, or in combination with any of the previous embodiments, the first circuit has a controlled volume and the second circuit has a variable volume.

In some embodiments, or in combination with any of the previous embodiments, the disinfectant assembly may further include a mixing member within the first circuit, wherein the mixing member is configured to mix the heated disinfectant liquid to increase the uniformity of the heated disinfectant liquid.

In some embodiments, or in combination with any of the previous embodiments, the mixing member may include a tortuous path mixing member that agitates the heated disinfectant liquid.

In some embodiments, or in combination with any of the previous embodiments, the apparatus may further include a probe positioning assembly having a securing member for securing the probe, one or more sensors, and a processor in electrical communication with the one or more sensors configured to receive a signal from the one or more sensors, wherein the one or more sensors are configured to capture indicator information from an indicator, wherein the indicator is operatively coupled to the probe, or wherein the one or more sensors are configured to capture presence information from the probe.

In some embodiments, or in combination with any of the previous embodiments, the indicator is operatively coupled to the cord of the probe.

In some embodiments, or in combination with any of the previous embodiments, the one or more sensors include a reflective object sensor and the indicator is a color, a hall-effect sensor and the indicator is a magnet, or a wireless identification reader and the indicator is a tag.

In some embodiments, or in combination with any of the previous embodiments, the one or more sensors is one or more indicator sensors including a first indicator sensor and the indicator information is captured from a first portion of the indicator, and wherein the signal is a first signal corresponding to the first portion of the indicator.

In some embodiments, or in combination with any of the previous embodiments, the one or more indicator sensors may further include a second indicator sensor and the indicator information is further captured from a second portion of the indicator, and wherein processor receives a second signal corresponding to the second portion of the indicator, and wherein the processor determines a position of the probe based on the indicator information captured from the first portion and second portion of the indicator.

In some embodiments, or in combination with any of the previous embodiments, the first sensor and the second sensor are reflective object sensors, wherein the first portion of the indicator is a non-reflective portion and the second portion of the indicator is a reflective portion, and wherein the processor is configured to determine that the probe is in a correct position as a result of a condition where the first signal indicates the reflective portion is detected by the first sensor and the second signal indicates the non-reflective portion is detected by the second sensor.

In some embodiments, or in combination with any of the previous embodiments, the one or more sensors is the presence sensor, and wherein the signal is an indication of the presence of the probe.

In some embodiments, or in combination with any of the previous embodiments, the presence sensor is a momentary push button switch.

In some embodiments, or in combination with any of the previous embodiments, the securing member may include an apparatus housing, one or more rollers, and one or more biasing members operatively coupled to the one or more rollers, wherein a cord of the probe is configured to be held by the one or more rollers and the housing through the force provided by the one or more biasing members.

In another aspect, the present disclosure embraces a method of disinfecting a probe. The method may include disinfecting the probe via at least one nozzle, the at least one nozzle applying a disinfectant heated by a heater assembly in a first circuit, and rinsing the probe with a rinse liquid heated by the heater assembly in a second circuit, wherein the first circuit and second circuit share a heating element from the heater assembly.

To the accomplishment the foregoing and the related ends, the one or more embodiments comprise the features hereinafter described and particularly pointed out in the claims. The following description and the annexed drawings set forth certain illustrative features of the one or more embodiments. These features are indicative, however, of but a few of the various ways in which the principles of various embodiments may be employed, and this description is intended to include all such embodiments and their equivalents.

### BRIEF DESCRIPTION OF THE DRAWINGS

Having thus described embodiments of the invention in general terms, reference will now be made to the accompanying drawings, where:

FIG. 1 illustrates a front view of a probe disinfecting apparatus, in accordance with some embodiments of the present disclosure;

FIG. 2 illustrates a front view of a probe disinfecting apparatus with an overlay of a portion of the internal comments, in accordance with some embodiments of the present disclosure;

FIG. 3 illustrates a schematic of the operation of dual heater of the probe disinfecting apparatus, in accordance with some embodiments of the present disclosure;

FIG. 4 illustrates a perspective view of the dual heater of the probe disinfecting apparatus, in accordance with some embodiments of the present disclosure;

FIG. 5 illustrates a perspective view of a mixing member for the dual heater of the probe disinfecting apparatus, in accordance with some embodiments of the present disclosure;

FIG. 6 illustrates a cross-sectional view of a probe reservoir assembly illustrating the depositing of the cleaner, disinfectant, and/or rinsing fluid on the probe, in accordance with some embodiments of the present disclosure;

FIG. 7 illustrates a perspective view of a probe disinfecting apparatus including a probe positioning system, in accordance with some embodiments of the present disclosure;

FIGS. 8A illustrates a side view of a probe positioning system, in accordance with some embodiments of the present disclosure;

FIG. 8B illustrates a side view of a probe positioning system, in accordance with some embodiments of the present disclosure;

FIG. 8C illustrates a side view of a probe positioning system, in accordance with some embodiments of the present disclosure;

FIG. 9 illustrates a side view of a probe positioning system, in accordance with some embodiments of the present disclosure;

FIG. 10 illustrates a side view of a probe positioning system, in accordance with some embodiments of the present disclosure;

FIG. 11A illustrates process flow for disinfecting a probe using the probe disinfecting apparatus, in accordance with some embodiments of the present disclosure; and

FIG. 11B illustrates a process flow for positioning a probe, in accordance with some embodiments of the present disclosure.

### DETAILED DESCRIPTION OF EMBODIMENTS OF THE INVENTION

Embodiments of the present invention now may be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure may satisfy applicable legal requirements. Like numbers refer to like elements throughout.

The apparatuses and methods of the present invention are described specifically with respect to cleaning and high-level disinfection of ultrasound probes (e.g., trans-vaginal, rectal, or the like probes); however, it should be understood that the apparatuses, the components therein (e.g., probe positioning system, dual heater, mixing member, or the like), and/or at a least a portion of the methods of the present invention may be utilized on any type of probe (e.g., TEE probes, or the like).

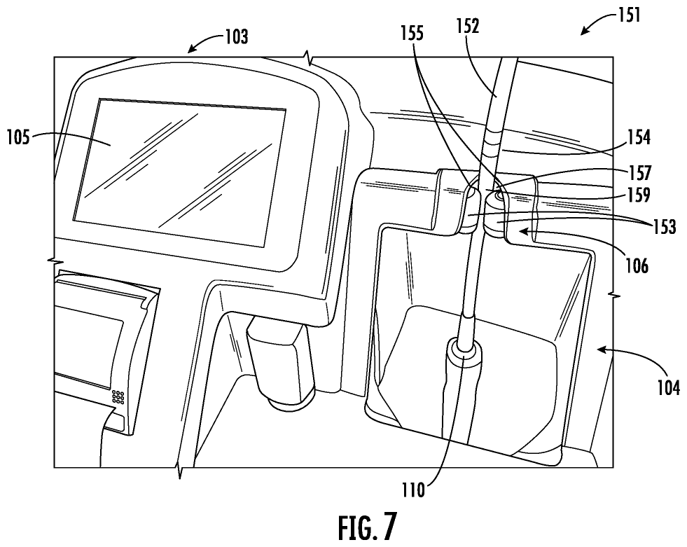

As will be described in further detail herein with respect to FIGS. 7 through 10, in some embodiments, the apparatuses and methods of the present invention may include a probe positioning system to provide an indication that (i) the probe has been placed in a securing member (otherwise described as a securing assembly, or the like) prior to the application of liquids thereto during cleaning, rinsing, and/or disinfecting, and/or (ii) that the probe is aligned (e.g., vertically, horizontally, rotationally, or the like) with the apparatuses providing the cleaning, rinsing, and/or disinfecting to the probe. The probe positioning system may utilize one or more sensors (including, but not limited to, reflective object sensors, hall effect sensors, and wireless readers such as RFID, Bluetooth, Wi-Fi, or the like). The one or more sensors may be operatively coupled to portions of the probe disinfecting apparatus proximate the probe, or to portions of the cord coupled to the probe. The probe, or the cord coupled thereto, may include targets (also referred to herein as “indicators”) corresponding to the type of sensor(s) utilized.

For example, in embodiments where one or more reflective object sensors are used, the target applied to the cord or probe may be an indicator of certain reflectiveness or color(s). In embodiments where one or more hall effect sensors are used, the target applied to the cord or probe may be a magnet. In embodiments where one or more wireless readers are used, the target applied to the cord or probe may be a tag.

Upon insertion of the probe or cord into the securing member, a presence sensor may provide a signal to portions of the control unit assembly (e.g., such as to the user interface, computer output, or various computer memory devices therein), to indicate to the control software that the probe has been inserted into the securing member. Additionally, the one or more sensors provide an indication signal to the control software as to whether the positioning of the probe is correct or incorrect (e.g., if the probe should be moved upwards or downwards in a vertical direction, laterally in a horizontal direction, rotated to change the orientation of the probe, or the like), which may provide an indicator on the user interface (e.g., control graphics of a graphical user interface, control light emitters, or control sound generators, or the like to provide assistance in positioning the probe).

The apparatuses and methods described herein may utilize a cleaner liquid (e.g., detergent, or the like) and/or rinsing liquid (e.g., water) to rinse the probes 110 before disinfecting to remove bioburden, soil, and the like (e.g., hemoglobin, carbohydrates, proteins, endotoxin, or the like) (described collectively herein as “foreign material”) from the probe after it is removed from a patient. As such, after the probe is positioned within the disinfecting apparatus, the disinfecting apparatus may be used to rinse, clean, and/or disinfect the probe. As will be described herein, the cleaner liquid and the rinsing liquid may be applied by one or more nozzles 108 depositing the liquids onto the probes. However, it should be further understood that any type of component may be used to apply the liquids onto the probes (e.g., submersion, misting, pouring, or the like the liquids). As such, the apparatus 100 may have a rinsing assembly that only rinses, a cleaning assembly that only cleans, or a rinsing and cleaning assembly that is used to clean and rinse the probe. As will be described herein, when a cleaner liquid is used, the cleaner liquid may be an enzymatic detergent that has bacteriostatic properties to inhibit bacterial growth in the apparatus 100 (e.g., within the rinsing assembly 124, supply lines, fluid circuit, or the like). The multiple enzymes in the cleaner liquid rapidly attack soils and have low foam properties for effective recirculation within the apparatus 100. If a cleaner liquid is used for cleaning the probe, the probe may be rinsed after cleaning to remove or substantially remove the residual cleaner liquid from the probe and the rest of the fluid circuit. If a cleaner liquid is not used, the probe may still be rinsed in order to aid in removing foreign material from the probe before disinfecting.

After cleaning and/or rinsing, a high-level disinfectant process is applied to the probe 110. The high-level disinfectant process deposits a disinfectant liquid on the probe 110 using the one or more nozzles 108 for a specified amount of time to disinfect the surface of the probe 110, and thereafter, the probe 110 is thoroughly rinsed to remove or substantially remove any remaining disinfectant liquid from the probe 110 or from within the rest of the fluid circuit. The patient never interacts with the apparatus 100, as such the apparatus 100 provides a layer of insulation between the patient and the cleaner and/or the disinfectant through both physical barriers as well as an air filter assembly.

The term cleaner liquid used herein may describe the cleaner in a form that is used to clean the probes 110. The cleaner liquid may be formed by mixing a concentrated dose (e.g., in solid or liquid form from a single or multi-dose container) with another liquid (e.g., water) or it may be received in a pre-mixed form (e.g., from a cleaner container or form storage outside of the apparatus 100). It should be understood that the use of the term cleaner liquid may be substituted with the term cleaner or cleaner solution throughout this application, and as such this specification may describe that the cleaner liquid (e.g., a concentrated dose and/or the cleaner mixed with water) may be utilized within the process steps or within the components of the apparatus 100 described herein. Likewise, the term disinfectant liquid used herein may describe the disinfectant in a form that is used to disinfect the probes. The disinfectant liquid may be formed by mixing a concentrated dose (e.g., in solid or liquid form from a single or multi-dose container) with another liquid (e.g., water) or it may be received in a pre-mixed form (e.g., from a disinfectant container or from storage outside of the apparatus 100). It should be understood that the use of the term disinfectant liquid may be substituted with the term disinfectant or disinfectant solution throughout this application, and as such this specification may describe that the disinfectant liquid itself (e.g., a concentrated dose and/or the disinfectant mixed with water) may be utilized within the process steps or within the components of the apparatus 100 described herein. Moreover, it should be understood that the term fluid circuit described herein may include the components and tubes within the apparatus in which the cleaner liquid, rinsing liquid, and/or disinfectant liquid passes through.

The present invention provides for the cleaning and/or disinfecting of a probe 110 within the apparatus 100. The apparatus 100 comprises a housing 101 that at least partially encloses the components of the apparatus 100, which both securely hold the probe 110 to avoid damage to the probe 110 and also control the processes for directing the rinsing liquid and/or cleaner liquid, and the disinfectant liquid through the flow paths of the apparatus 100 to clean and/or rinse and disinfect the probe 110.

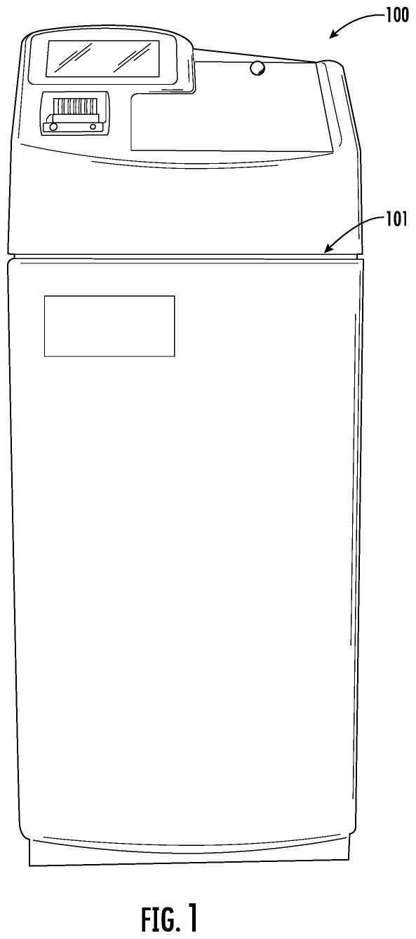

FIGS. 1-10 illustrate various views of the apparatus 100, or components thereof, in accordance with some embodiments of the present invention. The apparatus 100 houses and supports the probe (e.g., when inserted into the apparatus 100) within a probe reservoir assembly 102, such as through the use of the probe positioning system, as well as other components used to store, move, heat, quench, or the like, the disinfectant liquid, the cleaner liquid and/or rinsing liquid.

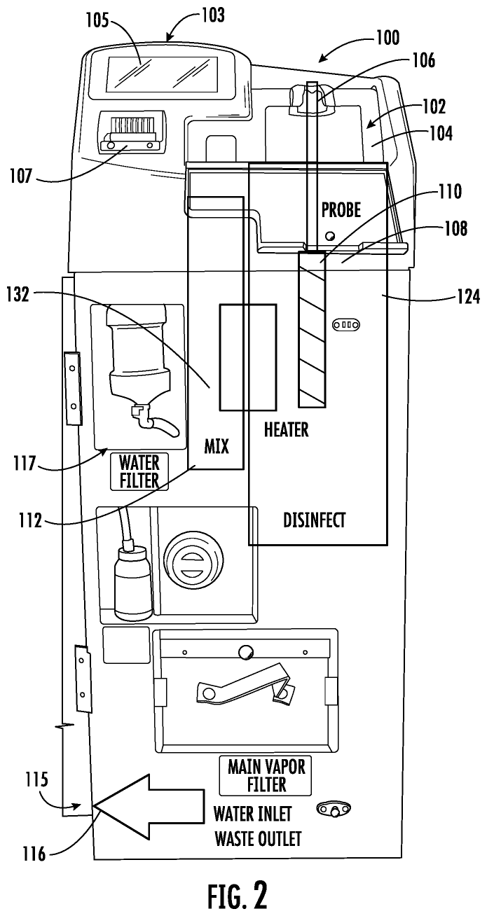

As illustrated in FIG. 2, the probe reservoir assembly 102 includes a probe reservoir 104, structured to hold the probe 110 and to contain and collect the rinsing liquid, cleaner liquid (e.g., if used), and/or the disinfectant liquid after it is applied to the probe 110. Within the probe reservoir assembly 104 is a securing member 106 (e.g., a holder, clamp, clip, hook, other fastener, or the like), the securing member 106 may be structured to grip, clamp, restrict movement of the probe 110 during the cleaning and/or rinsing and disinfecting process described herein.

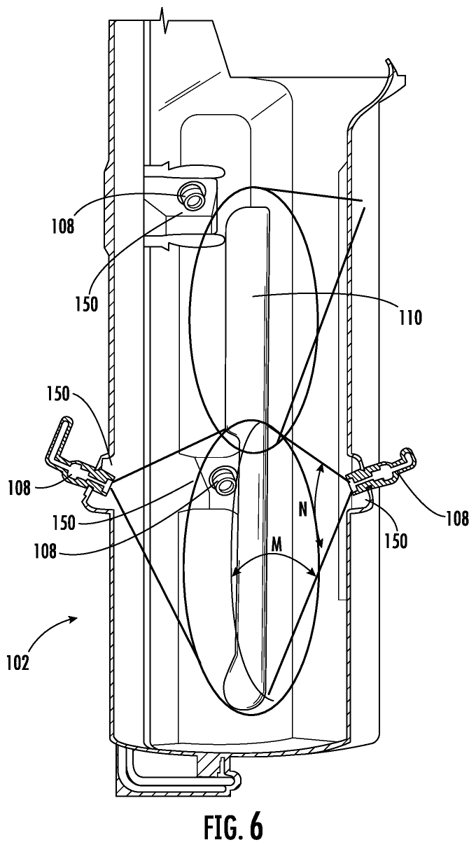

Referring now to FIG. 6, one or more nozzles 108 are positioned, at least partially, within the reservoir assembly 102, and are structured to apply (otherwise described as deposit, or the like) the disinfectant liquid, rinsing liquid, and/or cleaner liquid onto the probe 110. Each of the one or more nozzles 108 includes at least one aperture for dispensing the disinfectant liquid, rinsing liquid, and/or cleaner liquid onto the probe 110. In some embodiments, in order to minimize the number of nozzles 108 and/or to increase the coverage of the nozzles 108 applying the liquid onto the probe 110 (e.g., which generally has a longitudinal shape), the application pattern of the nozzles may be approximately an oval conical dispensing pattern (e.g., elongated cone). The application pattern of the nozzles 108 may be defined by a lateral application angle, M, and a longitudinal application angle, N. In some embodiments, the lateral application angle M may be approximately 60 degrees (e.g., range between 50, 52, 54, 56, 58, 60, 62, 64, 66, 68, 70, or the like degrees), while the longitudinal application angle N may be approximately 90 degrees (e.g., range between 80, 82, 84, 86, 88, 90, 92, 94, 96, 98, 100, or the like degrees). In other embodiments, various other lateral application and longitudinal application angles M, N of nozzles 108 may be used, such as to maximize and optimize the coverage of the application pattern of the nozzles 108 to various embodiments of probe 110 and/or reservoir assembly 102. As such, the angles of M, N may fall outside of and/or overlap any of the values described above.

The nozzles 108 may be positioned and spaced from the probe 110 such that each of the oval conical spray patterns from each of the one or more nozzles 108 overlaps with the oval conical dispensing pattern from an adjacent (e.g., nearby) nozzle 108, such that coverage of the disinfectant liquid, rinsing liquid, and/or cleaner liquid overlaps on the surfaces of probe 110, thereby reducing or eliminating portions of the probe 110 that are not subjected to the disinfectant liquid, rinsing liquid, and/or cleaner liquid. Accordingly, each of the nozzles 108 may be positioned within a corresponding receptacle 150 of the reservoir assembly, where each of the receptacles 150 contains an angled wall portion to which a nozzle 108 is mounted normally thereto. The angled wall portions of receptacles 150 may be structurally positioned to facilitate the proper angle of each corresponding nozzle 108 relative the probe 110 to ensure optimal coverage. The dispensing pattern of the nozzles 108 reduces the amount of liquid that does not contact the probe 110 (e.g., that misses being dispensed onto the probe). Once dispensed onto the probe 110, the dispensing liquid, rinsing liquid, and/or cleaner liquid will run down the surfaces of the probe 110 and collect at the bottom of the reservoir 104. While the spray pattern is generally described as an oval conical spray pattern, or other types of spray patterns may be used to minimize the liquid that misses the probe during application of the liquids. Moreover, while FIG. 6 illustrates a cross-sectional view of a reservoir assembly 102 having seven (7) nozzles 108 (e.g., three upper nozzles and four lower nozzles, some of which are not shown graphically in FIG. 6 as a result of the cross-sectional view), it shall be appreciated that, in other embodiments, fewer nozzles 108 or more nozzles 108 may be used. As such, the reservoir assembly 102 may have 2, 3, 4, 5, 6, 7, 8 upper nozzles and/or 2, 3, 4, 5, 6, 7, 8 or the like lower nozzles. The number of nozzles may range between, overlap, or fall outside of any of these values.

While the apparatus 100 in the figures is illustrated as having a single reservoir assembly, in other embodiments there may be multiple probe reservoir assemblies 102, or multiple probes may be fit within the probe reservoir assembly 102, in order to clean and/or disinfect multiple probes at once. Moreover, the one or more nozzles 108 are described as being stationary nozzles, but having dispensing patterns that may cover probes 110 of different sizes (e.g., widths, lengths, uniform, or non-uniform shapes). However, in alternate embodiments the one or more nozzles 108 may be moveable such that the direction of dispensing may be changed and/or adjusted such that the dispensing patterns may be changed. In other embodiments the apertures of the nozzles 108 and/or the nozzles themselves may be changed in order to change the dispensing direction and/or pattern.

The one or more nozzles 108 may be configured to apply the disinfectant liquid, rinsing liquid, and/or cleaner liquid in a uniform pattern such as to provide ample coverage of the disinfectant liquid, rinsing liquid, and/or cleaner liquid across the probe 110, such that the liquids will run down the surfaces of the probe 110 through the use of gravity. In this way, it may be necessary to reduce the velocity of the dispensing of the liquid for optimal application in order to reduce the amount of disinfectant liquid, rinsing liquid, and/or cleaner liquid that fails to maintain contact with the surfaces of the probe 110. However, in other embodiments it may be beneficial to dispense the liquids at higher velocities in order to remove foreign material from the probe 110.

While in some embodiments, the nozzles 108 are able to dispense the liquids on all of the surfaces of the probe 110, in the event that not all of the surfaces of probe 110 are located within the dispensing patterns of the one or more nozzles 108, the positioning of the probe 110 within the probe reservoir 104 (e.g., when secured by the securing member 106) is such that gravitational forces allow for the disinfectant liquid, rinsing liquid, and/or cleaner liquid to propagate downwards over the surfaces of the probe 110, including any surfaces that potentially did not directly receive liquid from the nozzles 108. Accordingly, the gravitational forces similarly allow for the disinfectant liquid, rinsing liquid, and/or cleaner liquid to cover the surfaces of the probe 110 and accumulate into a drainage accumulation section of the probe reservoir 104. This accumulation section is operatively coupled to a waste outlet (e.g., drain), and conduit therebetween, which enables the disinfectant liquid, rinsing liquid, and/or cleaner liquid collected at the accumulation section to exit the apparatus 100 for storage, disposal, or recycling.

As further illustrated in FIGS. 2 and 7, the apparatus 100 may comprise a control unit assembly 103 for housing the electronic components. The control unit assembly 103 may comprise a user interface 105 (e.g., graphical user interface, other interface, or the like) that is utilized to control the apparatus 100 and the electrical components for running the user interface 105. In some embodiments the user interface 105 may comprise a touchscreen and/or keypad, a scanner (e.g., barcode scanner, QR code scanner, digital image scanner, infrared scanner, or other like scanner), and an output device 107 (e.g., printer, output interface, connection to computer system, touchscreen, or the like). The touchscreen 105 and/or the keypad enable the user to initiate operation of the unit. The scanner allows the user to input data to the apparatus 100, for example data related to the cleaner liquid, disinfectant liquid, probe, user, process durations and/or temperatures, or the like. The output device 107, such as the printer, provides the user with a diagnostic output of the machine condition, operating status, success or failure of the cleaning and disinfecting of the probe, and/or the process parameters (e.g., temperatures, wet/dry conditions, durations of the steps of the process, or the like) of the cleaning, rinsing, and/or disinfecting process. For example, the output may include the confirmation of the position of the probe from the probe positioning system, temperatures from the first and second temperature sensors 146, 148, wet/dry indications from the wet/dry sensors, a filter replacement notification, duration of the cleaning, rinsing, disinfecting, and/or final rinsing process steps, or the like. It should be understood that in other embodiments of the present invention, the control unit assembly 103 may be combined with other assemblies, separated into two or more other assemblies, or configured in other ways in order to allow the user to control the apparatus 100 and receive output regarding the cleaning and disinfecting of the probe (e.g., through a user computing device, such as a mobile device, such as a smartphone or the like). It shall also be understood that the electronic control circuits referred to herein may be operatively coupled to the control unit assembly 103 and/or integrated entirely into the control unit assembly 103. Indeed, computer instructions executed by the processing device(s) of the control unit assembly 103 may result in the one or more electronic control circuits being implemented and performing the functions described herein, as discrete components of integrated circuits within the control unit assembly 103.

The apparatus 100 may comprise a water inlet and drain assembly 115. The water inlet and drain assembly 115 may comprise a water inlet 116, one or more drains, and a material trap (e.g., lint, debris, or other foreign material). The water inlet 116 provides a location to receive water and provide the water to the water filter assembly 117, which is discussed in further detail below. The drains allow for the removal of used cleaner waste, disinfectant waste, and/or rinsing waste (e.g., cleaner rinsing waste or disinfecting rinsing waste) that are flushed through the system. The one or more drains may include a specific drain for disinfectant waste and disinfecting rinsing waste, and a specific drain for cleaner waste and cleaner rinsing waste. The material trap is used to catch and separate the foreign material (e.g., bioburden, soil, and other biological material) from and the cleaner liquid, disinfectant liquid, rinsing liquid and/or waste (e.g., cleaner waste, disinfectant waste, cleaner rinsing waste, disinfecting rinsing waste, or the like) received from the probe reservoir 104 as the probe is cleaned, rinsed, disinfected, and final rinsed. For example, the material trap may be used to remove the foreign material from the cleaner liquid, rinsing liquid, disinfectant liquid, and/or waste as they are drained out of the system after completion of the cleaning, rinsing (e.g., cleaner rinsing, or pre-disinfecting rinsing), disinfecting, or final rinsing (e.g., disinfecting rinsing) steps. The material trap may be emptied as necessary, and the used cleaner waste, disinfectant waste, and/or water waste may be disposed of or recycled as needed. It should be understood that in other embodiments of the present invention the water inlet and drain assembly 115 may be combined with other assemblies, separated into two or more other assemblies, or configured in other ways in other embodiments of the invention in order to provide the source of water to the apparatus 100 and to remove the waste products of cleaning, rinsing, disinfecting, and final rinsing from the apparatus 100. In some embodiments, the water inlet and drain assembly 115 (or another assembly) may be operatively coupled to (e.g., contain, be connected to, or the like) a water supply tank that houses at least a portion of the water for use as the water supply for mixing with the cleaner or disinfectant, and/or used as the rinse liquid. In still other embodiments, one or more waste tanks may be operatively coupled to the drain assembly (or another assembly). The one or more waste tanks may be utilized to hold the waste from the cleaner and/or cleaner rinse cycles, and/or the disinfectant and/or disinfectant rinse cycles. The waste may be held within the one or more waste tanks while the waste material is inactivated to protect the public waste system. This embodiment may be particularly useful for the disinfectant and/or disinfectant rinse liquid, which may be required to be inactivated before disposing of the waste.

The apparatus further comprises a water filter assembly 117, as illustrated in FIG. 2. The water filter assembly 117 may comprise a water filter housing, a water filter, and water tubes and valves that receive water from the water inlet and drain assembly 115 and delivers the water directly or indirectly to the probe reservoir assembly 102, or other assemblies as discussed herein. The water filter may be a 0.2-micron water filter that filters water from particles that are greater than 0.2 microns. In other embodiments of the invention the water filter may be configured to filter particles that are less than or greater than 0.2 microns. It should be understood that in other embodiments of the present invention the water filter assembly 117 may be combined with other assemblies, separated into two or more other assemblies, or configured in other ways in order to supply the water for mixing with the cleaner to create the cleaner liquid, for mixing with the disinfectant to create the disinfectant liquid, and/or for rinsing the probe and/or the fluid circuit before and after cleaning and/or disinfecting.

The apparatus 100 may further comprise an air filter assembly. The air filter assembly may comprise an air filter duct, an air filter housing, an air filter, and a fan. The fan may pull any fumes from the cleaner, and more particularly from the disinfectant, away from the user located near the apparatus 100, and especially near the insertion point of the probe 110 in the probe reservoir assembly 102. As such, the air filter duct may be operatively coupled to the probe assembly reservoir 102, the rinsing assembly 124, the disinfectant assembly 112, and/or generally within the housing of the apparatus 100. The air filter duct may be operatively coupled to the fan, for example through the air filter housing and/or the air filter, in order to draw the fumes into the air filter housing through the air filter to remove or reduce potentially dangerous components from the fumes, and to exhaust the air out of the apparatus 100 through the fan. In some embodiments, the air filter is a carbon air filter that absorbs the peracetic acid (“PAA”) (or other components) in the fumes of the disinfectant. However, in other embodiments the air filter may be any type of filter that removes or reduces potentially dangerous components from the fumes of the cleaner and/or the disinfectant. It should be understood that in other embodiments, the air filter assembly may be combined with other assemblies, separated into one or more other assemblies, or configured in other ways in order to remove fumes from the apparatus 100. It should be understood that the air filter may comprise an RFID tag, 1D or 2D barcode, QR code, or other indication illustrating the type of filter that may be used to remove the fumes form the cleaner and/or disinfectant. In other embodiments, detector sensors may be utilized to indicate when the filter has reached, or is about to reach, its end of useful life. The detector sensors may be operatively coupled to the control unit assembly 103, and thus, the user may be notified when the filter has reached, or is about to reach, its end of useful life.

The housing 101 of the apparatus may comprise various features to access the components of the apparatus. In one embodiment the housing 101 may include a housing door that may be utilized to access the air filter assembly for accessing and replacing the air filter, and to access the water filter assembly 117 for accessing and replacing the water filter. In another embodiment, the housing 101 may include a housing service panel that allows a user to access the one or more assemblies contained within the housing.

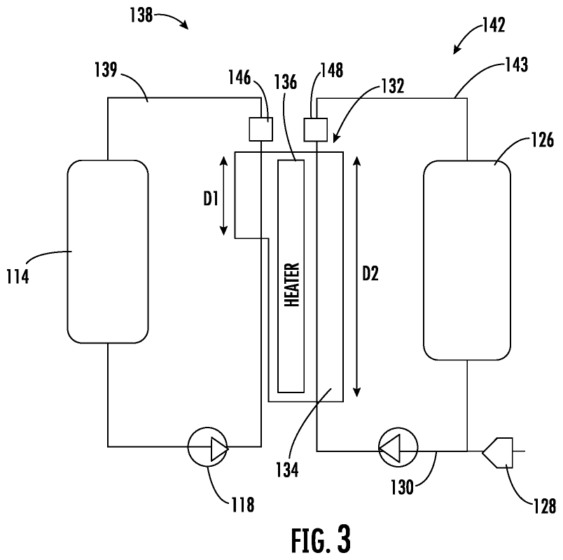

As generally discussed with respect the various assemblies described herein, each assembly that transfers rinsing liquid (e.g., water), cleaner liquid, disinfectant liquid, a mixture of these fluids, or the like may utilize supply and return lines (e.g., tubing) to operatively couple the assemblies together and to transfer the fluids from one assembly to another. The assemblies and the supply and return lines may be described herein as the fluid circuit. The supply and return lines may be as illustrated in one embodiment, at least in part in FIG. 3.

FIG. 3 illustrates a diagram of a portion of a fluid flow system of the apparatus 100, in accordance with an embodiment of the invention, and in particular, when utilizing the dual heater (also referred to as a “singular” or “singular body” heater) described herein. In the illustrated embodiment of FIG. 3, the circuits include the disinfectant circuit and the rinsing circuit. However, it should be understood that in other embodiments the rinsing circuit and/or the disinfecting circuit may also be used as a cleaning circuit. Alternatively, the fluid circuit may further include a cleaner circuit (e.g., a third circuit), and the heater may be a tri-heater.

Regardless of the configuration, it may be advantageous for heat to be applied to disinfectant liquid, rinsing liquid, and/or cleaner liquid prior to application to the probe 110. In some embodiments of the present invention the disinfectant liquid may be formed by mixing a concentrated dose (e.g., solid or liquid dose) of disinfectant with water and heating the disinfectant liquid. The addition of heat to a solvent (e.g., water or other solvents) and/or concentrated disinfectant (e.g., granular, concentrated liquid, dry powder, or the like) allows for the efficient mixing and dissolving of the concentrated disinfectant into the solvent. In this way, larger volumes of disinfectant can be readily created within the apparatus 100 by supplying the concentrated disinfectant, via a disinfectant supply, to the apparatus and continuously or periodically circulating the solvent and concentrated disinfectant solution while applying heat thereto.

Accordingly, in the first circuit 138 of FIG. 3, concentrated disinfectant may be applied to the disinfectant assembly 112 via a disinfectant supply. The disinfectant assembly 112 may comprise a disinfectant reservoir 114, a disinfectant cover, and a means for puncturing (e.g., projection, punch, blade, scissor, or the like) a disinfectant container containing the disinfectant (e.g., the concentrated dose, or the like). The disinfectant reservoir 114 is configured for receiving the disinfectant container (e.g., a one-time use bottle, package, or the like), which provides the disinfectant for mixing with the water to form the disinfectant liquid, and for delivery to the probe 110 via one or more nozzles 108 for disinfecting the probe 110. The disinfectant liquid is utilized to remove, neutralize, or otherwise sterilize the external surfaces of the probe that may have residual foreign material (e.g., residual bioburden, soil, and other like biological material) that is left on the surface of the probe 110 after the initial cleaning process.

In some embodiments, the concentrated disinfectant may be introduced to a portion of the first circuit 138, such as through a valve or pump, or directly into the disinfectant reservoir 114. In other embodiments, the user may insert an unopened single use disinfectant container into the disinfectant reservoir 114 of the disinfectant assembly 112 (e.g., concentrated dose, pre-mixed dose, or the like). The single use disinfectant container may be inserted at the beginning of the process, or in some embodiments may be inserted after a cleaning and/or rinsing process is completed. The dosage of the disinfectant within the container may be pre-determined (e.g., in a concentrated dose, or pre-mixed) in order to remove the need to test the disinfectant in a reservoir before each disinfecting step in order to identify if the disinfectant has the desired potency. However, in other embodiments of the invention, the container of disinfectant may be a multi-use bottle that the disinfectant assembly 112 may regulate in order to apply the desired amount to the probe assembly 102.

The first circuit 138 comprises the first conduit 139, which may be made of any plastic, composite, alloy, or metal tubing or rigid piping, including but not limited to: PVC, CPVC, Acetal, Nylon, EVA, ETFE, PEEK, PFA, polycarbonate, polyethylene, polypropylene, PTFE, Teflon®, nylon, copper, aluminum, braided hose, stainless steel, and so forth. The first conduit 139 may be defined by a substantially circular cross-section with a void throughout the length of the first conduit 139, the void structured to transport liquid from one end of the first conduit 139 to another end of the first conduit 139. It shall be appreciated that when referring to the first conduit 139 or second conduit 143, this description makes reference not only to individual sections of first and second conduit 143 (e.g., spliced sections of tubing connected by another feature of the first or second circuits 138, 142), but also to the continuous flow path of fluid(s) between any given two or more points within the first or second circuits 138, 142.

As illustrated in FIG. 3, the first conduit 139 may be operatively coupled to the disinfectant reservoir 114 and terminate at a first pump 118, the first pump 118 being any electro-mechanical device structured to move fluid through mechanical action, controlled by an electronic or hydraulic control device. The first conduit 139 continues at the exit of the first pump 138 and is routed to the first channel 140 of the heater 134 (e.g., dual-heater, or the like). As will be described in detail herein, heat is applied to the disinfectant within the void of the first channel 140, and the heated disinfectant exits the heater 134. A first temperature sensor 146 may be operatively coupled to the heater 134, adjacent the heater 134, within the first conduit 139, or otherwise within the first circuit 138 in order to determine the temperature of the heated disinfectant liquid. The first temperature sensor 146 outputs a temperature of the disinfectant liquid to provide an input to an electronic control circuit to change the output of the heater and/or to direct the flow rate of first pump 118 based on a temperature setpoint.

In some embodiments, the first temperature sensor 146 outputs a temperature of the disinfectant liquid to provide an input to an electronic control circuit to direct the voltage or electronic current supplied to the heating element 136 of heater 134 based on a temperature setpoint. The resistance of the heating element 136 transforms electronic energy into thermal energy (e.g., heat), the level of thermal energy is thus controlled by varying the voltage or electronic current supplied to the heating element 136. Thus, if the first temperature sensor 146 indicates a temperature of the disinfectant liquid lower than the temperature setpoint, the electronic control circuit may supply additional voltage or electronic current to the heating element 136 in order to increase the heat emitted by the heating element 136. Similarly, if the first temperature sensor 146 indicates a temperature of the disinfectant liquid is higher than the temperature setpoint, the electronic control circuit may reduce the voltage or electronic current to the heating element 136 in order to decrease the heat emitted by the heating element 136.

Alternatively, or additionally, if the first temperature sensor 146 indicates a temperature of the disinfectant liquid lower than the temperature setpoint, the function of the first pump 118 is lowered such as to lower the flow rate of the disinfectant liquid and increase the amount of time and given portion of disinfectant liquid is in contact with the heater 134. Similarly, if the first temperature sensor 146 indicates a temperature of the disinfectant liquid higher than the temperature setpoint, the function of the first pump 118 is raised such as to increase the flow rate of the disinfectant and decrease the amount of time and given portion of disinfectant liquid is in contact with the heater 134.

A segment of first conduit 139 further transports the disinfectant back to the disinfectant reservoir 114, to which the first conduit is operatively coupled. In this way, a “closed loop” system may be formed such that the disinfectant may be continuously heated and recirculated through the first circuit 138. The first circuit 138 may be supplied concentrated disinfectant via the disinfectant supply and/or disinfectant reservoir 114, and once a solvent is integrated into the first circuit 138, the first circuit 138 is activated via supplying electricity to the first pump 118, heating element 136, and/or first temperature sensor 146 and the concentrated disinfectant is dissolved or mixed with the solvent with the assistance of the added heat from the heating element 136 in heater 134 by increasing the molecular motion (e.g., kinetic energy) in the solution of concentrated disinfectant and solvent.

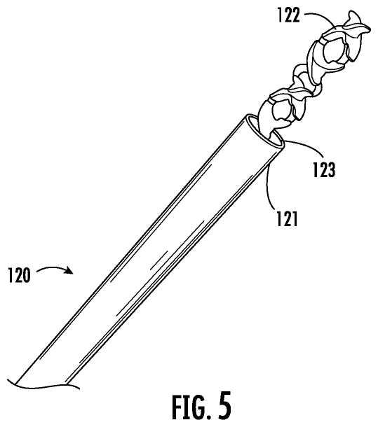

Referring now to FIG. 5, a portion of the first circuit 138 (e.g., the first conduit 139, the channel of the heater, or the like) may be configured with a mixing member 120. In some embodiments, the mixing member 120 is comprised of a mixing conduit 121, formed of any plastic, composite, alloy, or metal tubing or rigid piping, including but not limited to: PVC, CPVC, Acetal, Nylon, EVA, ETFE, PEEK, PFA, polycarbonate, polyethylene, polypropylene, PTFE, Teflon®, nylon, copper, aluminum, braided hose, stainless steel, and so forth. The mixing conduit 121 is defined by a substantially circular cross-section with a void 123 throughout the length of the mixing conduit 121, the void 123 structured to receive a tortuous path mixing member 122 and transport liquid from one end of the mixing conduit 121 to another end of the mixing conduit 121. In some embodiments, the mixing member 120 may be positioned within the first channel 140 of the heater 134, such as to mix and heat simultaneously. In other embodiments, the mixing member 120 may be positioned at any point within the first circuit 138, such as to be integrated within first conduit 139 (e.g., the mixing conduit 121 and the first conduit 139 are the same component), or in between sections of the first conduit 139.

The tortuous path mixing member 122, also known as a static mixing element, remains stationary within the void 123 and provides for the turbulent flow of the disinfectant solution within the void 123, which assists in the mixing or dissolving of the concentrated disinfectant with the solvent. The tortuous path mixing member 122 may be defined by a continuous helical element or double helical element, and may be comprised of any number of rigid materials such as stainless steel, copper, or various plastic of sufficient rigidity so as to not deform under stresses from the flow and/or heat of the disinfectant through the mixing member 120. The combined effect of dissolving or mixing the concentrated disinfectant and the solvent with the mixing member 120 and the added heat from the heater 134 is greater than the effect of implementing mixing member 120 or heater 134 alone.

The apparatus 100 may further comprise the rinsing assembly 124, as illustrated in FIG. 2-3. As previously discussed herein, the apparatus 100 illustrated may not utilize a cleaner liquid in addition to the rinsing liquid. However, in other embodiments in which a cleaner is utilized, the rinsing assembly 124 may include a cleaner supply (e.g., a cleaner pump, or other like means for distributing the cleaner solution). The illustrated rinsing assembly 124 comprises a rinsing reservoir 126 and the second circuit 142. The rinsing liquid (e.g., heated) is used to remove the foreign material from the probe before it is disinfected using the disinfectant liquid. The rinsing liquid removes material from the surface of the probe to allow the disinfectant liquid in the process step described herein to disinfect the surface of the probe that may contain residual foreign material that cannot be seen by the naked eye. After the disinfecting step, the probe may be thoroughly rinsed using the rinsing liquid (e.g., heated or unheated water) for 10-12 minutes, to remove the disinfectant solution from the probe and the probe reservoir 104. In other embodiments of the invention, the probe may be rinsed for another amount of time (e.g., for 2, 4, 6, 8, 10, 12, 14, 16, 18, or the like minutes) or may range within a duration that is inside of this range, outside of this range, or overlaps this range.

Referring back now to FIG. 3, the same heater assembly 132 (e.g., dual heater assembly) and corresponding heater 134 are used to supply heat to the second circuit 142 (e.g., the rinsing liquid and/or the cleaning liquid depending on the configuration of the apparatus 100). It shall be appreciated that a heated rinse and/or cleaner is more effective at rinsing and/or cleaning the probe 110, and accordingly heat should be applied to the rinsing liquid or cleaner liquid in the second circuit 142 before applying the rinsing liquid or cleaner liquid to the probe 110.

While traditional probe disinfecting apparatuses use a plurality of heaters to control the temperatures of various circuits (e.g., a first heater for the disinfecting liquid and a second heater for the rinsing liquid and/or cleaner liquid), the apparatus 100 described herein improves upon such traditional designs by incorporating a plurality of circuits into a singular heater with a singular heating element. In this way, the manufacturing and operation costs are significantly reduced. Further, by more than one circuit sharing a heater, fewer replacement parts are necessary to consider in maintaining the apparatus 100.

The rinsing liquid and/or cleaner liquid may be stored or temporarily housed in a rinsing reservoir 126. The second conduit 143 may be operatively coupled to the rinsing reservoir 126 and terminate at a second pump 130, the second pump 130 being any electro-mechanical device structured to move fluid through mechanical action, controlled by an electronic or hydraulic control device. The second conduit 143 continues at the exit of the second pump 130 and is routed to the second channel 144 of the heater 134. Heat is applied to the rinsing liquid and/or cleaner liquid within the void of the second channel 144, and the heated rinsing liquid and/or cleaner liquid exits the heater 134. A second temperature sensor 148 may be operatively coupled to the heater 134, adjacent the heater 134, within the second conduit 143, or otherwise within the second circuit 142 in order to determine the temperature of the heated rinsing liquid (or the heated cleaner liquid). The second temperature sensor 148 outputs a temperature of the rinsing liquid to provide an input to an electronic control circuit that adds additional rinsing liquid (e.g., at a lower temperature) to control the temperature of the rinsing liquid.

For example, prior to any rinsing and/or cleaning of the probe 110, the electronic control circuit opens the water inlet 128 to allow water to flow into the second circuit 142. The water entering the second circuit 142 is provided by a water source (e.g., a water supply) with a temperature lower than that of the rinsing liquid in the second circuit 142. By opening the water inlet 128, the electronic control circuit allows the pressurized water from water supply to enter the second circuit 142 and thereby quench the rinsing liquid to a lower temperature. Before or during the rinsing and/or cleaning process(es), the second temperature sensor 148 may read the temperature of the rinsing liquid and/or cleaner liquid and provide a continuous or intermittent temperature measurement to the electronic control circuit. Once a designated temperature setpoint has been reached, the water inlet 128 may thereafter be closed to prevent any further cooling of the rinsing liquid and/or cleaner liquid. Alternatively, or additionally, as previously described with respect to the first circuit 138, the flow (e.g., speed) of the rinsing liquid through the heater may adjusted to change the temperature of the rinsing liquid.

A segment of second conduit 143 is operatively coupled to the second channel 144, which further transports the rinsing liquid (or the cleaner liquid in alternate embodiments) back to the rinsing reservoir 126, to which the second conduit 143 is operatively coupled. In the embodiments in which a cleaner is utilized with the rinsing assembly 124 or in a separate assembly, the cleaner may be supplied directly or indirectly to the tubing (e.g., conduit) of the second circuit 142 to the rinsing reservoir 126, or to a separate circuit to a separate cleaner reservoir. As previously discussed, and like the disinfectant, the cleaner may be a concentrated dose that mixed with the water in the second circuit 142 or in another circuit, or it may be a pre-mixed ready to use dose. The cleaner may be supplied in a single use container, a multi-use container, from tank, or the like as previously discussed with the supply of the disinfectant. The cleaner liquid, like the rinsing liquid may be heated, applied to the probe, and recycled through the system one or more times (e.g., a single time, or multiple times).

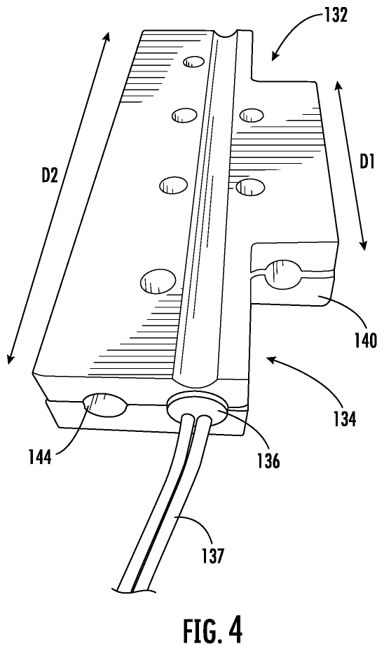

The heater assembly 132 described herein may be structured in different ways in order to operate as previously described herein. However, FIG. 4 illustrates the heater assembly 132 in accordance with some embodiments of the present disclosure. The heater assembly 132 may be comprised of a heater 134, which may be formed from a heat conducting material such as stainless steel, steel, aluminum, alloy, or other like metal or heat conducting material. The heater 134 is structured to receive a heating element 136, such as a cartridge heater, ceramic heater, or the like, which contacts the heater 134 and allows for heat to dissipate through the heater 134 through conductive heating. The heating element 136 may be received through a heater aperture within the heater 134. The heating element 136 is structured with conductors 137 which receive and transmit electrical energy to the heating element 136, the current of the electrical energy creating heat through the resistance of the material of the heating element 136.

The heater 134 may further be structured with at least two apertures, the first channel 140 and the second channel 144, which extend though the body of the heater 134 and are operatively coupled to the first and second conduits 139, 143. In some embodiments, the first channel 140 and the second channel 144 receive the first and second conduits 139, 143. In other embodiments, the first and second channels 140, 144 may be configured with connectors (e.g., adaptors, fittings, or the like) configured to receive the first and second conduits 139, 143, such that the first and second conduits 139, 143 do not extend through the heater 134, rather the first and second conduits 139, 143 terminate at the connectors and/or adapters and the first and second channels 140, 144 serve to transmit the disinfectant liquid, rinsing liquid, and/or cleaner liquid through the heater 134. Moreover, as illustrated in FIG. 4, the heater 134 may also be structured with one or more fastener apertures and threaded portions to assist with the assembly of the heater 134, which may be fabricated in two or more portions that may be operatively coupled together. In some embodiments the two portions of the heater 134 may be mirror images of the other.

Various probes 110 may contain components that are sensitive to extreme temperatures, such as temperatures of the disinfecting liquid and the rinsing liquid and/or cleaning liquid above temperature setpoints prescribed by the manufacturer of the probe 110. Alternatively, certain temperature may be required to provide proper disinfecting and rinsing and/or cleaning of the probes. As a result, the quenching (e.g., rapid cooling) of the rinsing liquid (or in other embodiments the cleaner liquid) may be necessary by the introduction of water to the second circuit 142 via the water inlet 128 to bring the rinsing liquid (or in other embodiments the cleaner liquid) to the prescribed temperature(s).

Different methods may be used to heat the disinfectant liquid and/or the rinsing liquid (or the cleaner liquid) in different ways. For example, in some embodiments, as illustrated in FIGS. 3 and 4, the position of the first and second channels 140, 144 in relation to the geometry of the heater 134 is such that the first channel 140 is defined by a length D1, whereas the second channel 144 is defined by a length D2. In some embodiments, the length of D1 is less than half of D2. In this way, the amount of heat supplied to the first channel 140 may be less than half of that of the second channel 144. This heat differential between the first and second channels 140, 144 is a result of the surface area exposed to the disinfectant in the first channel 140 compared to the larger surface area exposed to the rinsing liquid and/or cleaner liquid in the second channel 144. Due to the larger surface area of D2 relative to that of D1 (e.g., due to the longer length of D2), the rinsing and/or cleaner liquid will be subjected to the heat from the heating element 136 for a longer period of time as the corresponding liquids flow through the first and second channels 140, 144.

Although FIG. 4 illustrates a length difference between D1 and D2, it is contemplated that in some embodiments a similar objective of a heat differential between the first and second channels 140, 144 may be achieved where D1 and D2 are of equal length (or similar lengths), and instead the diameter of the apertures defined by the first and second channels 140, 144 are dissimilar. For example, the diameter of the second channel 144 may be larger than that of the first channel 140, such that the surface area exposed to the rinsing liquid and/or cleaning liquid in the second channel 144 is larger than that of the disinfectant at the first channel 140.

Alternatively, or additionally, in some embodiments, the function of the second pump 130 may be lowered if the second temperature sensor 148 indicates a temperature of the rinsing liquid and/or cleaner liquid is lower than the temperature setpoint. Accordingly, the lower flow rate of the rinsing liquid and/or cleaner liquid increases the amount of time a given portion of rinsing liquid and/or cleaner liquid is in contact with the heater 134, and thereby increase the temperature of the rinsing liquid and/or cleaner. Similarly, if the second temperature sensor 148 indicates a temperature of the rinsing liquid and/or cleaner liquid is higher than the temperature setpoint, the water inlet 128 is opened such that water is added to the second circuit 142, thereby reducing the temperature of the rinsing liquid and/or cleaner liquid in the second circuit 142.

Software monitors the cleaning and disinfecting process using first and second temperature sensors 146, 148 and/or wet/dry sensors. As illustrated by the schematic diagram in FIG. 3, first and second temperature sensors 146, 148 may be located in, or at the exit of, the heater assembly 132. In other embodiments of the invention, first and second temperature sensors 146, 148 may be located in other areas of the apparatus 100, or additional temperature sensors may be utilized in other areas. The first and second temperature sensors 146, 148 may provide information that allows the software to determine if the rinsing liquid (e.g., water), cleaner liquid, and/or disinfectant liquid is heated to the desired temperatures for the cleaning, rinsing, disinfecting, and final rinsing processes. The wet/dry sensors may be located at the inlet to the heater assembly 132, at the inlet to the probe reservoir assembly 102, or in the probe reservoir 104. In other embodiments of the invention the wet/dry sensors may be located in other areas of the apparatus 100. The wet/dry sensors may provide information that allows the software to determine if the liquid (e.g., water, cleaner, or disinfectant) is or is not located in the desired areas during different times of the cleaning and disinfecting process. As such, the software may provide information regarding the temperatures, and the locations and duration that the fluids are present within the apparatus 100 to the user through the output device 107 (e.g., printed, displayed in an interface, or the like). Moreover, the software may indicate whether or not the probe passed the cleaning, rinsing, disinfecting, and final rinsing process steps without encountering any errors.

FIG. 7 illustrates a perspective view of a probe disinfecting apparatus 100 including a probe positioning system 151, in accordance with some embodiments of the invention. As previously described, inconsistencies in the positioning of the probe 110 in a location relative the one or more nozzles 108 or any other disinfecting apparatus may alter the efficiency and/or effectiveness of the disinfecting process. Accordingly, maintaining the probe 110 securely in the probe disinfecting apparatus 100 while also allowing for adjustment to the positioning of the probe 110 improves the efficiency and/or effectiveness of the disinfecting process. The embodiment illustrated in FIG. 7 includes a securing member 106 that includes two rollers 153, each roller 153 disposed from the opposing roller 153 at a distance sufficiently small enough to prevent the cord 152 from being inadvertently dislodged from receptacle 159. While much of the disclosure described in detail herein refers to the cord 152 and being secured by the securing member 106, it shall be appreciated that in some embodiments of the invention the securing member 106 may instead secure the probe 110 in the receptacle 159 instead of the cord 152.

Upon presenting the cord 152 to the rollers 153 and pushing the cord 152 into the gap between the rollers 153, the rollers retract via one or more biasing members 155 (e.g., springs, leaf springs, spring arms, or the like) coupled to each of the rollers 153, widening the gap between the rollers 153. The spring arms 155 are attached to the housing 101 of the probe disinfecting apparatus, while each roller 153 is attached to a distal end of a corresponding spring arm, with the rotating axes of each roller 153 positioned substantially vertical and substantially parallel to the longitudinal axis of the cord 152. As the rollers 153 retract, continued pushing of the cord 152 into the widening gap between the rollers 153 allows for the rotation of the rollers 153, which assist in the cord 152 passing through the gap between the rollers 152 and entering the receptacle 159. Once the cord 152 is in the receptacle 159, the springs of the spring arms 155 naturally return to a partially relaxed state, narrowing the gap between the rollers 153 while still providing spring force to retain the cord 152 in the receptacle 159 via the rollers 152. Put differently, the cord 152 is retained by pushing the cord 152 into the rollers 153, spreading the rollers 153 against the force of the spring arms 155 attached thereto, and then pushing the cord 152 past the centers of the rollers 155 such that the cord 152 is held on two sides by the rollers 153 and one side by the housing of the probe disinfecting apparatus itself. This method permits a wide range of cord diameters to be accommodated and held securely. In some embodiments it should be understood that a single roller 153 and biasing member 155 may be used to secure the cord 154, such as by holding the cord between the roller 153 and a housing 101 of the disinfecting apparatus 100.

Alternatively, as previously described herein, different securing members 106 may be utilized to secure the probe 110. For example, in other embodiments the securing member 106 that secures the probe 110 (or cord thereof) may be a hook, a clamp, actuating grip, the like that is able to hold the probe (or cord 154 thereof) in place, alone or through the use of a biasing member, or other component.

Regardless of the securing member 106 used, the probe positioning system 151 of the probe disinfecting apparatus 100 may also include a presence sensor 157 to determine whether a cord 152 or the probe 110 have been provided to the probe disinfecting apparatus 100 such that the probe disinfecting apparatus 100 may begin the disinfecting process. In some embodiments, the presence sensor 157 may be operatively coupled to the housing of the probe disinfecting apparatus 100 and positioned with respect to the securing member 106. For example, with respect to the roller configuration illustrated in FIG. 7, the presence sensor 157 may be located in the receptacle 159 between the rollers 153 and the housing of the probe disinfecting apparatus. As such, upon insertion of the cord 152 through the rollers 153 and into the receptacle 159, the presence sensor 157 may indicate to a control unit (e.g., send a signal, or the like) of the probe disinfecting apparatus 100 that the cord 152 (and, therefore, the probe 110) is secured in the probe reservoir 104 of the probe disinfecting apparatus 100.

In some embodiments, the presence sensor 157 may be a momentary push button switch, structured to close an electrical circuit in communication with the momentary push button switch upon the pressing of the push button, and structured to open the electrical circuit upon releasing the push button. Additionally, or alternatively, various other devices may be used as a presence sensor 157, including, but not limited to, capacitive touch sensors, proximity sensors, magnetic reed switches, Hall effect sensors, and/or infrared (“IR”) sensors.

In some embodiments, the presence sensor 157 may be positioned elsewhere in the probe disinfecting apparatus, such as above or below the securing member 106. Additionally, or alternatively, the presence sensor 157 may be positioned on the housing 101 of the probe disinfecting apparatus 100 to be proximate the probe 110 within the reservoir 104, and instead of providing affirmation of the presence of the cord 152, it may provide affirmation of the presence of the probe 110 itself.

The probe positioning system 151 of the probe disinfecting apparatus 100 may also include components to determine that the probe 110 is in the proper position for efficient probe cleaning, rinsing, and disinfecting. As such, the probe positioning system 151 may detect the position of the probe 110 directly through the location of the probe 110, or indirectly through the location of the cord 152, and provide feedback (e.g., to the user) of a determination as to whether the probe (e.g., directly or through the cord 152) is in the desired position. As a result of feedback from the probe positioning system, the position of the probe 110 or cord 152 may be adjusted. Such adjustments may occur by pushing or pulling the cord 152 upwards or downwards through the receptacle 159 while the cord 152 is being secured by the securing member 106 (e.g., in the receptacle 159 by the rollers 153, or other securing member 106). Additionally, or alternatively, the cord 152 may be removed from the securing member 106, such as from the receptacle 159 by expanding the gap between rollers 153, pulling the cord 152 through the gap, and repositioning the cord 152 within the securing member 106, such as by inserting the cord 152 through the gap between the rollers 153 and into the receptacle 159.

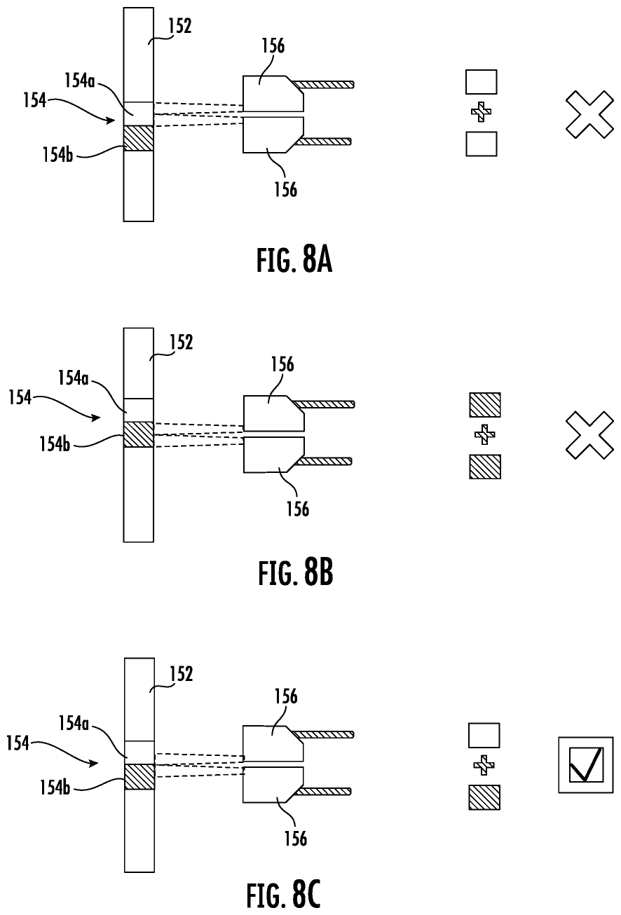

FIGS. 8A-8C illustrate side views of a probe positioning system, in accordance with some embodiments of the invention. In some embodiments, the cord 152 or probe 110 may include an indicator 154. The indicator 154 may be any type of indicator 154 formed from tape, tubing, paint, coating, label, or the like on the probe 110 or cord 152, or formed by the material, etched into the material, extending from the material, or the like of the probe or the cable, or combination thereof. Since different types of probes having different dimensions (e.g., length, width, or the like) may be used within the probe positioning system, the indicator 154 may be applied to different probes at different locations on the probes 110 and/or cord 152 based on an optimized position for the probes 110 within the probe reservoir 104 of the probe reservoir assembly 102. As such, in some embodiments the indicator 154 may be operatively coupled at different locations on a probe 110 (e.g., directly on the probe, or on a location of the cord 152) based on the type of probe 110 being disinfected.

In the embodiment illustrated in FIGS. 8A-8C, the indicator 154 is comprised of a first indicator portion 154a and a second indicator portion 154b (or otherwise described as two indicators 154). In some embodiments, the first indicator 154a and the second indicator 154b, may have a different attribute, such as a different color, material, pattern, shape, text, image, or the like. In some embodiments, the first indicator 154a may be a different material or color than the second indicator 154b. In the embodiment illustrated in FIGS. 8A-8C, the first indicator 154a is a reflective white color capable of being indicated by a reflective object sensor 156 as being reflective. The second indicator 154b is a non-reflective black color capable of being indicated by a reflective object sensor 156 as being non-reflective.

In some embodiments, the probe disinfecting apparatus 100 may include one or more reflective object sensors 156 that may be used in order to determine the presence or lack of presence of the indicator 154. In particular embodiments, two reflective object sensors 156 are oriented vertically relative one another and spaced apart at a predetermined distance based on the desired dimensions of the indicator 154. The reflective object sensors 156 in FIGS. 8A-8C are spaced apart such that the measurement area of one reflective object sensor 156 may receive the reflectiveness of the first indicator 154a, while the measurement area of the other reflective object sensor 156 may receive the reflectiveness of the second indicator 154b. The differences in the reflectiveness between the first and second indicators 154a, 154b allows for the determination of the probe 110 (directly or through the use of the cord 152) is positioned properly when one reflective object sensor 156 indicates a reflective object, while the other reflective object sensor 156 indicates a non-reflective object. A determination of reflectiveness may be made by comparing the output of each sensor to a predetermined threshold.

Accordingly, and as shown in FIG. 8A, both of the reflective object sensors 156 may receive reflections from the indicator 154 (or cord 152) at their corresponding measurement areas above the predetermined threshold. Therefore, since both of the measurement areas are deemed reflective, the probe 110 (directly or through the cord 152) are deemed to be not properly positioned in the vertical direction.

As shown in FIG. 8B, both of the reflective object sensors 156 may receive reflections from the indicator 154 (or cord 152) at their corresponding measurement areas below the predetermined threshold. Therefore, since both of the measurement areas are deemed non-reflective, the probe (directly or through the cord 152) are deemed to be not properly positioned in the vertical direction.

However, as shown in FIG. 8C, one reflective object sensor 156 may receive a reflection from the indicator 154 (or cord 152) at their corresponding measurement areas that indicate one measurement area being above the predetermined threshold, while the other measurement area being below the predetermined threshold. Therefore, the indicator 154 is aligned properly in relation to the reflective object sensors 156, and the probe 110 (directly or through the cord 152) are deemed to be positioned properly in the vertical direction.

While the probe positioning system described herein has been generally described with respect to the locating the probe 110 vertically within the disinfectant apparatus 100, as previously described herein, alternatively, or additionally, the probe positioning system may be utilized to determine the lateral and/or rotational position of the probe 110. For example, if the securing member 106 allows for installation of one or more probes 110 in different lateral positions and/or since the probe 110 may have a non-uniform shape, it may be beneficial to confirm the lateral and/or the rotational orientation of the probe 110 (e.g., directly or through the use of the cord 152). As such, additionally, or alternatively, the one or more indicators 154 and/or the one or more sensors 156 may be used to determine the lateral position and/or rotational orientation of the probe 110 in the same or similar way as described with respect to the determination of the vertical position of the probe 110.

In some embodiments, the output of the presence sensor 157 may be used in conjunction with the output of the reflective object sensors 156 to both confirm the presence of the probe 110 and the position of the probe 110 (directly or through the use of the cord 152), such as to eliminate unnecessary position measurements or the inadvertent initiation of cleaning, rinsing, or disinfecting cycles. In some embodiments, if one reflective object sensor 156 indicates a reflectiveness above a predetermined threshold, while the other reflective object sensor 156 indicates a reflectiveness below a predetermined threshold, if the presence sensor 157 does not indicate the presence of the probe 110 (directly or through the use of the cord 152), the status of the probe disinfecting apparatus 100 may default to a condition where no cleaning, rinsing, or disinfecting occurs due to lack of probe 110. Additionally, or alternatively, the output of the presence sensor 157 may chronologically precede the output of the reflective object sensors 156 such as to not measure the indicator 154 using the reflective object sensors 156 and instead set the status of the probe disinfecting apparatus 100 to a default condition where no cleaning, rinsing, or disinfecting occurs due to lack of probe 110. Additionally, or alternatively, the presence sensor 154 and the reflective object sensors 156 may periodically provide signals at a predetermined interval (such as time intervals or at various stages of the cleaning, rinsing, or disinfecting cycles) to ensure that the probe 110 or cord 152 remains in the desired position throughout the cleaning, rinsing, or disinfecting cycle.

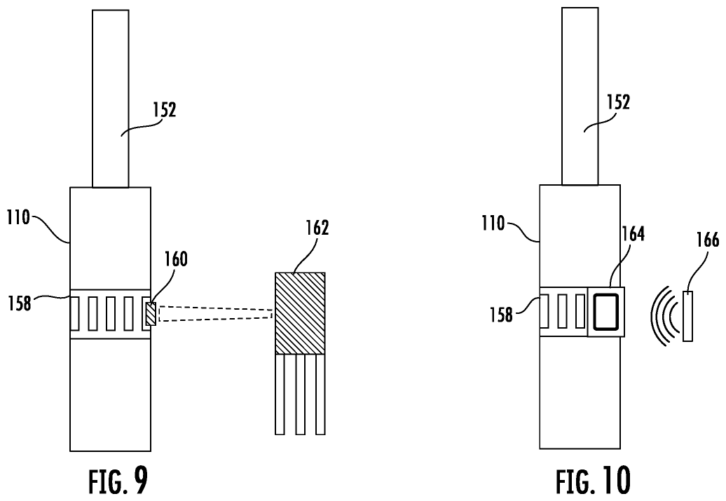

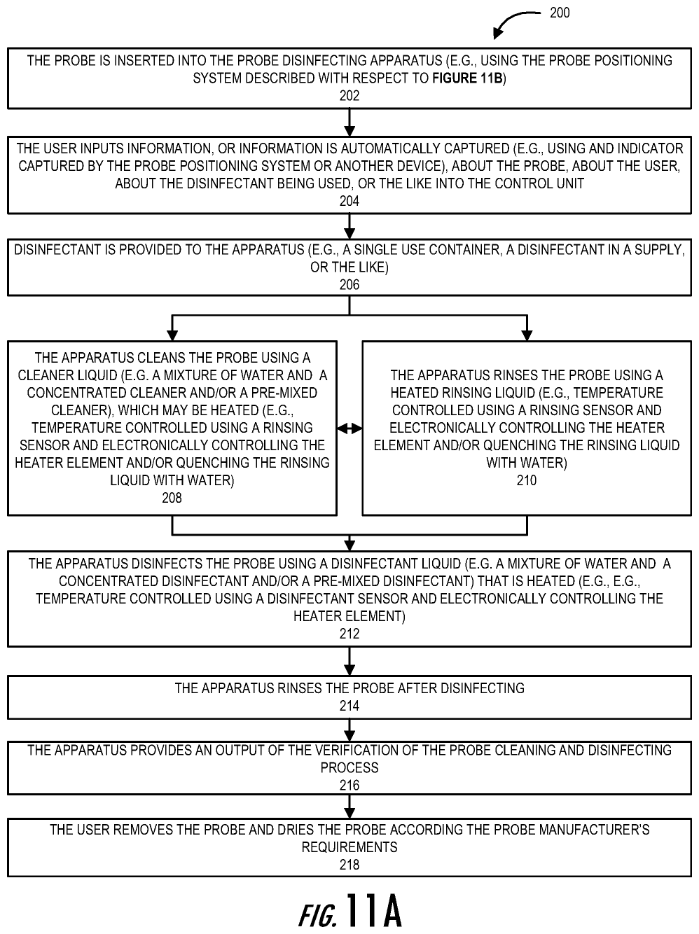

FIGS. 9 and 10 illustrate side views of a probe positioning system 151, in accordance with some embodiments of the invention. While much of the preceding disclosure is described with respect to reflective object sensors 156, in other embodiments the probe positioning system 151 may use alternative devices to provide the indication signals. In one such embodiment, as shown in FIG. 9, the sensor may be a Hall effect sensor 162 that is used to sense an indicator that is a magnet 160 that is operatively coupled to the probe 110 (e.g., directly or through the cord 152), such as through the use of a mount 158 (e.g., peripheral mount, or the like). Examples of mount 158 include, but are not limited to, hose clamps, bands, belts, fabricated brackets, fastener(s), beads of glues or epoxies, or any other method to secure the magnet to the periphery of the probe 110 or cord 152. When the probe 110 is incorrectly positioned, the Hall effect sensor 162 does not sense the magnetic field of the magnet 160 and may provide an indication signal accordingly. When the probe 110 is correctly positioned, the Hall effect sensor 162 senses the magnetic field of the magnet 160 and may provide an indication signal accordingly.

In another embodiment, as shown in FIG. 10, a wireless reader 166 may be implemented to sense a tag 164, another alternate indicator, operatively coupled to the probe 110 by the mount 158. In some embodiments, the wireless reader 166 may be an RFID reader, while the tag 164 is an RFID tag. In some embodiments, the wireless reader 166 may be a Near-Field Communication (“NFC”) reader, while the tag 164 is an NFC tag. In some embodiments, the wireless reader 166 may be a Bluetooth Low Energy (BLE) beacon and a corresponding BLE tag 164. Regardless of the specific technology and components used, when the probe 110 is incorrectly positioned, the wireless reader 166 does not sense the tag 164 and may provide an indication signal accordingly. When the probe 110 is correctly positioned, the wireless reader 166 senses the tag 164 and may provide an indication signal accordingly.

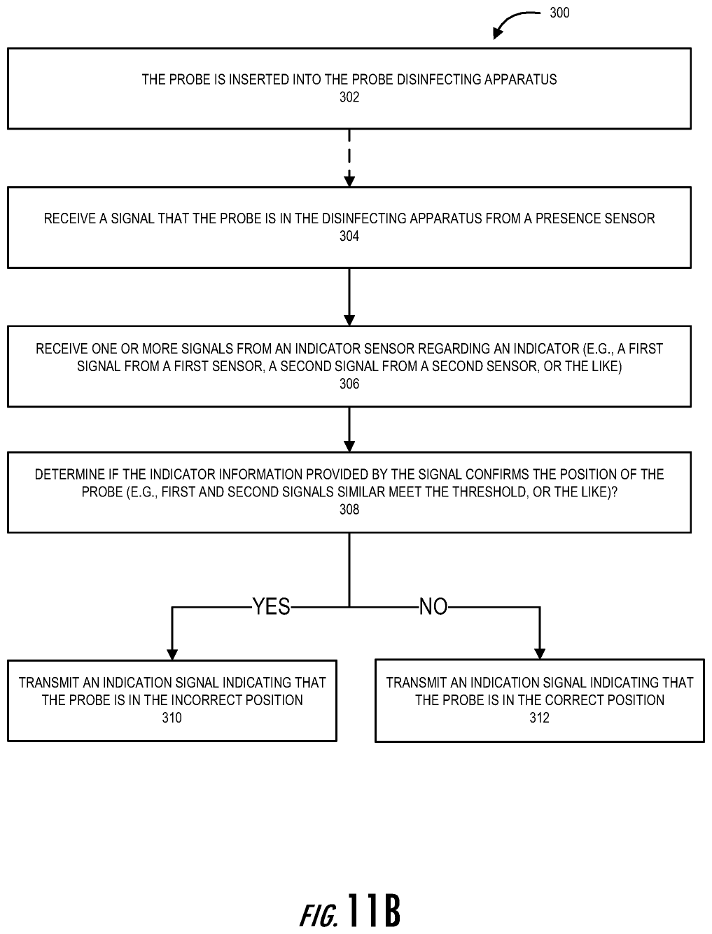

FIG. 11A illustrates a high-level process flow for disinfecting and rinsing and/or cleaning a probe 110 using the positioning system and/or a dual heater assembly 132 described herein. As illustrated by block 202 a soiled probe (e.g., which may have been manually or automatically pre-cleaned) may be inserted into the apparatus 100. As previously described, the probe may be secured within a probe reservoir assembly 102 with a securing member 106 (e.g., through a holder, clamp, clip, hook, other fastener, or the like) to restrict the movement of the probe. Although FIG. 11A may be described with respect to a single probe, in some embodiments of the invention the apparatus 100 may be able to accommodate multiple probes at a time, for example within one or more probe reservoir assemblies 102 and/or securing members 106. As described herein, a probe position system may be used to position the probe 110 within the disinfectant apparatus 100, as described with respect to the process illustrated in FIG. 11B.

As such, FIG. 11B illustrates a process flow 300 for positioning a probe 110, in accordance with some embodiments of the invention. At block 302, the probe 110 is inserted into the securing member 106 of the probe disinfecting apparatus 100. For example, as discussed herein the cord 152 may be captured in a receptacle 159 defined by the space between two rollers 153 and the housing of the probe disinfecting apparatus 100, where the cord 152 is held in place by forces exerted on the cord 152 by the rollers 153, the rollers having force applied to them by spring arms 155.

At block 304, a signal may be received from a presence sensor 157, which may be positioned in the receptacle 159, indicating that the probe 110 (directly or through the cord 152) is within the securing member 106.

Additionally, or alternatively, at block 306, one or more signals may be received from an indicator regarding detection or non-detection of an indicator on the probe 110 (or cord 154 thereof). For example, a first signal may be received from a first reflective object sensor 156, wherein the first signal corresponds to the reflectiveness of a first portion of an indicator 154 measured by a first reflective object sensor 156 (e.g., a first measurement indicator). The signal may then be compared to a predetermined threshold to determine whether the reflectiveness of the first portion is above or below the predetermined threshold. Additionally, or alternatively, a second signal may be received from a second reflective object sensor 156, wherein the second signal corresponds to the reflectiveness of a second portion of an indicator 154 measured by a second reflective object sensor 156 (e.g., the second measurement area). The signal may then be compared to a predetermined threshold to determine whether the reflectiveness of the second portion is above or below the predetermined threshold. As previously described, the first and second portions of the marker 154 measured are vertically disposed from one another as a result of the first and second reflective object sensors 156 being vertically disposed from one another. It should be understood that additionally, or alternatively additional signal may be received regarding a lateral and/or rotational position of the probe 110, as previously discussed herein.

At decision block 308, a determination is made if the signal from the indicator sensor confirms that the indicator 154 detected is in the correct location and/or orientation. As discussed herein, in some embodiment the signal provides indicator information that is compared to stored threshold information by the probe positioning system in order to determine if the probe 110 is in the correct position.

As illustrated in block 310, an incorrect indication signal may be sent that the probe 110 is in the incorrect position when the indication information confirms the probe 110 is not located in the correct position. For example, as previously discussed herein, signals (indicating the reflectiveness) provided from the first and second reflective object sensors 156 may be compared to one another. If the signals are the same or similar, for example, both the first and second portions of the marker measured are determined to be reflective or both the first and second portions of the marker measured are determined to be non-reflective (e.g., based on a predetermined threshold), then an indication signal is transmitted that indicates that the probe 110 is in the incorrect position.

As illustrated in block 312, a correct indication signal may be sent that the probe 110 is in the correct position when the indication information confirms the probe 110 is located in the correct position. For example, if one of the first or second portions of the indicator 154 measured is determined to be reflecting, while the other of the first or second portions of the indicator 154 is determined to be non-reflective, then an indication signal is transmitted that indicates that the probe 110 is in the correct position.

In some embodiments, the indication signal is transformed into computerized graphic objects and displayed on a user interface 105 to display to an operator an indication that the probe 110 is either correctly positioned or incorrectly positioned. For example, the computerized graphic objects may be “check” or “X” marks, warnings, words or other text, color indicators, directional arrows, or a graphical depiction of the probe 110, and so forth, or any combination thereof.

Additionally, or alternatively, the indication signal may induce a light emitter (such as LED lighting) to illuminate, turn off, or strobe to indicate that the probe 100 is either correctly positioned (for example, by illuminating a green LED) or incorrectly positioned (for example, by illuminating a red or orange LED).

Additionally, or alternatively, the indication signal induces a sound generator, such as a speaker, to emit a sound to indicate if the probe is positioned correctly or incorrectly. The sound generator may emit a first tone to indicate that the probe 110 is correctly positioned, and a second tone to indicate that the probe 110 is incorrectly positioned. Additionally, or alternatively, the tones from the sound generator may be emitted at different rates to indicate that the probe 110 is either correctly positioned (for example, by emitting fixed-duration tones at rapid intervals) or incorrectly positioned (for example, by emitting fixed-duration tones at longer intervals). Additionally, or alternatively, the sounds may include a voice indication that indicates correct or incorrect positioning.

Additionally, or alternatively, the indication signal may induce an automated positioning member (e.g., robotic arm, or the like) to position the probe 110 in a predetermined vertical direction or along a predetermined path.

Returning to FIG. 11A, after positioning as illustrated by block 204, a user inputs information into the apparatus 100 through a control unit assembly 103, which is described in further detail herein. The information may be related to the probe being cleaned, rinsed, and/or disinfected, the user operating the apparatus 100, the disinfectant liquid, rinsing liquid, and/or cleaner liquid being used to disinfect, rinse, and/or clean the probe, the duration of time and/or temperatures for cleaning, rinsing, disinfecting, final rinsing, or other process steps. In some embodiments, these programmed times for cleaning, rinsing, disinfecting, final rinsing, and/or other like process steps are pre-programmed into the apparatus 100. As such, in some embodiments when the processing temperatures and/or times are pre-programed, the user does not have the ability to change these inputs. If the pre-programed process is not followed or the cycle is interrupted before completion, the cycle may be aborted and a failure notice may be provided to the user (e.g., a failure ticket is printed, a notification displayed on an interface, or the like). In some embodiments, these operating parameter inputs may be automatically determined based on the probe positioning system described above. For example, the probe positioning system may identify other information from the indicator 154, such as the type of probe being disinfected, and automatically set the operating parameter inputs of the disinfectant apparatus 100 based on the information captured by the probe positioning system. After the inputs are set, the cleaning, rising, disinfecting, and/or final rinsing steps of the process may begin.

As illustrated in block 206 of FIG. 11A, disinfectant is provided to the disinfecting apparatus 100. In some embodiments, a predetermined amount of disinfectant is loaded into the apparatus 100. For example, in some embodiments of the invention the disinfectant is a single use container (e.g., bottle, package, or the like for a concentrated dose, pre-mixed dose, etc.) that is utilized once and the container is disposed of after the disinfecting step is completed. The single use container may be punctured inside of the apparatus 100 to contain and minimize splashes, spills, and vapors within the apparatus 100. In other embodiments of the invention, the disinfectant may be delivered from a disinfectant supply that has more than a single use, and that is loaded into the apparatus 100 or located externally to the apparatus 100. In some embodiments of the invention the single use container and/or the dosage from a multi-use container may be utilized without the need to add additional liquid or water (e.g., a ready to use dose), while in other embodiments the single use container and/or the dosage from a multi-use container (e.g., a concentrated dose) may be mixed with liquid or water to form a disinfectant liquid that is used to disinfect the probe.

The water used herein for creating a cleaner liquid, a disinfectant liquid, or for rinsing liquid may be 0.2-micron filtered bacteria free water. In other embodiments of the invention the water used to create the cleaner liquid, the disinfectant liquid, or rinsing liquid, may be water that is less than or greater than the 0.2 micron filtered bacteria free water.

At this step of the process, both the first and second circuits 138, 142 may be filled with their respective liquids (e.g., disinfectant liquid and rinsing liquid, and in some embodiments the cleaner liquid). Accordingly, before, during, or after the first and second pumps 118 and 130 are engaged, electricity is supplied to the heating element 136 of the heater 134 to generate heat. Accordingly, both of the corresponding liquids in the first and second circuits 138, 142 may be simultaneously heated by the same heater 134.

Block 208 of FIG. 11A illustrates that in some embodiments the apparatus 100 cleans the probe by applying a pre-determined amount of cleaner liquid, dispensed via one or more nozzles 108 to the probe from the rinsing assembly 124, as has been described in detail herein. The cleaner liquid may be supplied as a dose from a multi-use supply (e.g., 50 or more or less doses within a container, or other like dose amount), or as a dose from a single use supply (e.g., single use container). In some embodiments of the invention, the single use container and/or the dosage from a multi-use container may be utilized without the need to add additional water (e.g., a ready to use dose), while in other embodiments the single use container and/or the dosage from a multi-use container (e.g., a concentrated dose) may be mixed with water to form a cleaner liquid in order to disinfect the probe. In some embodiments the cleaner is mixed with filtered water from the water filter assembly 117 to create the cleaner liquid before being used to clean the probe.

Moreover, in some embodiments the cleaner liquid is heated. As previously described in detail, as a result of the heating process using the heater 134, the cleaner liquid may or may not be heated above a temperature conducive to cleaning the probe 110 without causing damage to fragile components of the probe 110. As such, in the event the temperature is too high, the electronic control circuit may open water inlet 128 to allow water to enter the second circuit 142, thereby quenching the cleaner liquid to approximately 40 degrees C. In other embodiments, the cleaner liquid may be quenched to a temperature in the range of 35 degrees C. to 45 degrees C., inclusive. However, it should be understood that the temperature to which the cleaner liquid is quenched may be within this range, overlap this range, or fall outside of this range in alternate embodiments of the invention (e.g., range between 30, 32, 34, 36, 38, 40, 42, 44, 46, 48, 50, or the like degrees C.).

In some embodiments of the invention, the cleaner liquid may be dispensed onto the probe (e.g., dispensed onto the probe by one or more nozzles and recirculated) for a minimum of five (5) minutes to remove the foreign material from the surface of the probe. In other embodiments, the cleaner liquid may be dispensed onto the probe for less than or greater than five (5) minutes (e.g., 2, 2.5, 3, 3.5, 4, 4.5, 5, 5.5, 6, 6.5, 7, 7.5, 8, 8.5, or the like minutes or range between, overlap, or fall outside any of these values). Once dispensed onto the probe 110, the cleaner liquid will run down the surfaces of the probe 110 through gravity and collect at the bottom of the reservoir 104.

The cleaner liquid (e.g., the heated cleaner solution) is dispensed to the probe 110 via one or more nozzles, and thereafter recirculated back through the fluid circuit to the probe 110 again in one or more cleaner cycles to clean the probe before the probe 110 is disinfected. The heater 134 may continuously heat the cleaner liquid as it is recycled through the fluid circuit in order to maintain or increase the temperature of the cleaner liquid at or to the desired temperature range. Accordingly, the electronic control circuit may similarly once again open water inlet 128 to quench the cleaner fluid if the temperature of the cleaner fluid is above the temperature setpoint, as will be indicated to the electronic control circuit by the second temperature sensor 148.

Each use of the cleaner liquid may be a single use, and thus, the cleaner liquid waste may be discarded to the one or more drains after a cleaning cycle. In some embodiments the cleaner liquid may have a dedicated cleaner drain to keep the cleaner liquid waste from mixing with the disinfecting liquid waste. Moreover, in some embodiments a material trap may be used to remove the foreign material from the cleaner liquid (e.g., cleaner solution) during each cycle of the cleaner liquid through the fluid circuit or after the cleaning step is complete. In some embodiments, the cleaner liquid may be delivered to the probe 110 and discharged from the apparatus in one or more cycles (e.g., a single cycle), and thereafter, a new second cleaner liquid (e.g., the heated cleaner solution) may be delivered to the probe 110 in a second cycle, and so on (e.g., third cleaner delivered in a third cycle, or the like).

It some embodiments, the cleaner liquid may not be utilized. As such, after, before, during, or in lieu of the cleaning cycle described in block 208, the apparatus 100 rinses the probe 110 using a rinsing liquid (e.g., heated rinsing liquid) via dispensing the rinsing liquid onto one or more nozzles 108, as illustrated in block 210. When cleaning liquid is used, after cleaning, the probe (via one or more nozzles 108), as well as the components and tubes of the fluid circuit, may be thoroughly rinsed by rinsing fluid in one or more rinsing cycles (e.g., cleaner rinsing cycles). In some embodiments, after each rinsing cycle, the rinsing waste (e.g., cleaner rinsing waste) is also discarded to the one or more drains, such as the dedicated cleaner drain. The water used to rinse the probe 110 may also be heated in some embodiments of the invention (e.g., to the same or similar temperatures as described with respect to the cleaner and/or the disinfectant). During the rinsing step the water may be recycled through the fluid circuit, or new water may be used within each cycle of the cleaner rinsing step.

Regardless of when the rising occurs (e.g., with or without a cleaning cycle), the rinsing liquid may be heated prior to being dispensed to the probe 110 by passing through the portion of second circuit 142 containing the heater assembly 132 using the heater 134, with the temperature of the rinsing liquid controlled using the second temperature sensor 148 (e.g., the rinsing sensor). As previously described, the second temperature sensor 148 may be operatively coupled to the heater 134, adjacent the heater 134, within the second conduit 143, or otherwise within the second circuit 142 in order to determine the temperature of the rinsing liquid. As a result of the heating process using heater 134, the rinsing liquid may or may not be heated above a temperature conducive to rinsing the probe 110 without causing damage to components of the probe 110 (e.g., shell of the probe or the like). Accordingly, when the temperature is too high, the electronic control circuit may open the water inlet 128 to allow water to enter the second circuit 142, thereby quenching the rinsing liquid to approximately 40 degrees C. In other embodiments, the rinsing liquid may be quenched to a temperature in the range of 35 degrees C. to 45 degrees C., inclusive. However, it should be understood that the temperature to which the rinsing liquid is quenched may be within this range, overlap this range, or fall outside of this range in alternate embodiments of the invention (e.g., range between 30, 32, 34, 36, 38, 40, 42, 44, 46, 48, 50, or the like degrees C., or the like).

In some embodiments, the amount of heat applied to the rinsing liquid may be adjusted by controlling the voltage or electronic current to the heating element 136 of the heater 134. Additionally, or alternatively, the heat applied to the rinsing liquid may be adjusted by varying speed at which the rinsing liquid passes adjacent the heating element 136 by adjusting the speed of the second pump 130.

Block 212 of FIG. 7 illustrates that the apparatus 100 disinfects the probe using a disinfectant liquid that is heated. The apparatus heats the high-level disinfectant liquid to approximately 38-40 degrees C., inclusive, before being dispensed onto the probe 110 via one or more nozzles 108 for disinfecting. In other embodiments the disinfectant liquid may be heated to another range that falls outside, overlaps, or is located within this range.

The disinfectant liquid may be a mixture of a solvent (e.g., water) and a concentrated disinfected (e.g., concentrated dose), or in some embodiments the disinfectant may be a pre-mixed disinfectant (e.g., a pre-mixed dose). In embodiments where a concentrated disinfectant is supplied to the first circuit 138, the solvent may be heated before being combined with the concentrated disinfectant, or the concentrated disinfectant and solvent mixture may be heater after or during the combination of concentrated disinfectant and solvent.

The concentrated disinfectant, solvent, or mixture thereof form a disinfectant liquid that is heated in the first circuit 138 by electronically controlling the heating element 136 (in the heater assembly 132) to which the disinfectant liquid is exposed in the first channel 140. By varying the electrical voltage or current to the heating element 136, the amount of heat generated by the heating element 136 is proportionately varied. Thus, the temperature of the disinfectant liquid is subsequently varied as a result of the contact between the disinfectant liquid and the heater 134. In some embodiments, the temperature of the disinfectant liquid may also be varied by varying the speed at which the disinfectant liquid passes adjacent the heating element 136 by adjusting the speed of the first pump 118 or adjusting the amount of heat applied to the disinfectant liquid by varying the heat of the heating element 136.

In some embodiments, the disinfectant liquid (e.g., disinfectant solution) may be dispensed onto the probe 110 by the one or more nozzles 108 and recirculated back through the system to the probe 110 during one or more disinfectant cycles to disinfect the probe. The disinfectant liquid may be heated each time it is recirculated through the fluid circuit (e.g., first circuit 138) in order to maintain the temperature of the disinfectant liquid to the desired temperature range. In some embodiments, the disinfectant liquid may comprise PAA, and make up 2.65% of the disinfectant liquid, with the remainder comprising of water and/or other components. In other embodiments, a different type of disinfectant liquid may be utilized and/or the amount of the concentration of disinfectant may be below or above the recited 2.65% relative to the solvent (e.g., 2.25, 2.35, 2.45, 2.55, 2.65, 2.75, 2.85, 2.95, 3.05, or the like percent or range between, overlap, or fall outside of these values). The disinfectant liquid may be dispensed by one or more nozzles 108 (e.g., delivered and recycled) to the probe 110 for at least three (3) minutes at the desired temperature range. In other embodiments of the invention the minimum amount of application time via one or more nozzles 108 time may be below or above the at least three (3) minutes (e.g., 2.25, 2.5, 2.75, 3, 3.25, 3.5, 3.75, 4, or the like minutes, or range between, overlap, or fall outside of these values). In some embodiments, after disinfecting the probe 110, the disinfectant liquid (e.g., disinfectant solution) may have a dedicated disinfectant drain in order to keep the disinfectant waste from mixing with the rinsing liquid and/or cleaner waste.

In some embodiments, the disinfectant liquid may be dispensed by one or more nozzles to the probe 110 and thereafter discharged from the apparatus in one cycle (e.g., a single cycle). In some embodiments, a new second disinfectant liquid (e.g., the heated disinfectant solution) may be delivered to the probe 110 in a second cycle, and so on (e.g., third disinfectant liquid delivered in a third cycle, or the like). The disinfectant liquid waste may be required to be kept separate from the cleaner waste (and other rinsing liquid waste) because the disinfectant waste may have to be chemically inactivated, depending on the requirements of different facilities or areas of use.

In some embodiments of the present invention, since the disinfectant liquid used with each cycle is received from a single use disinfectant container, no monitoring of the disinfectant liquid's potency is required, nor is there any requirement for daily testing of the disinfectant liquid. The single use containers are created with the desired potency, and as such no measurement of the disinfectant liquid is needed before it is utilized for disinfection.

As was the case with the cleaner step, in some embodiments a material trap may be used to remove the foreign material from the disinfectant liquid (e.g., disinfectant solution) or rinsing liquid during each cycle of the disinfectant liquid or rinsing liquid, or after the disinfectant step or rinsing steps are complete.

As illustrated in block 214, the apparatus 100 may repeat the rinsing process as described in block 210, or in some embodiments if the rinsing step in block 210 is forgone, the rinsing step in block 214 is performed. As such, during the disinfecting step of block 212, additional rinsing liquid may be passing through the second circuit for rinsing after the disinfecting. As such, during the disinfecting step additional rinsing liquid may be heated (or rinsing liquid may be continuously heated in the second circuit). In some embodiments, the rinsing step as described in block 210 may be heated, while the rinsing liquid as described with respect to block 214 may not be heated. In embodiments where the rinsing liquid is heated in block 214, prior to being dispensed to the probe 110, the rinsing liquid passes through the portion of second circuit 142 containing the heater assembly 132 using the heater 134, with the temperature of the rinsing liquid controlled using the second temperature sensor 148 (e.g., the rinsing sensor). The second temperature sensor 148 may be operatively coupled to the heater 134, adjacent the heater 134, within the second conduit 143, or otherwise within the second circuit 142 in order to determine the temperature of the rinsing liquid. As a result of the heating process using heater 134, the rinsing liquid may be heated above a temperature conducive to rinsing the probe 110 without causing damage to fragile components of the probe 110. Accordingly, the electronic control circuit may open the water inlet 128 to allow water to enter the second circuit 142, thereby quenching the rinsing liquid to approximately 40 degrees C. In other embodiments, the rinsing liquid may be quenched to a temperature in the range of 35 degrees C. to 45 degrees C., inclusive. However, it should be understood that the temperature to which the rinsing liquid is quenched may be within this range, overlap this range, or fall outside of this range in alternate embodiments of the invention.

In some embodiments, the amount of heat applied to the rinsing liquid may be adjusted by controlling the voltage or electronic current to the heating element 136 of the heater 134. Additionally, or alternatively, the heat applied to the rinsing liquid may be adjusted by varying speed at which the rinsing liquid passes adjacent the heating element 136 by adjusting the speed of the second pump 130.

Block 216 of FIG. 7 illustrates that the apparatus 100 may provide output of the verification of the cleaning, rinsing, and/or disinfecting of the probe 110. The verification of the cleaning and disinfecting of the probe 110 may be based on the use of sensors that measure temperature (e.g., first and second temperature sensors 146, 148), wet/dry areas of the apparatus 100 (e.g., wet/dry sensors), or other like process or apparatus parameters. For example, the apparatus 100 may provide the results of diagnostic tests that confirm that the probe 110 has been properly disinfected because the probe was cleaned and/or rinsed, disinfected, and rinsed again with corresponding liquids for the desired durations and at the desired temperatures. The output verification may occur in the form of a printed document. However, in other embodiments of the invention, the output may be displayed on a screen that is operatively coupled to the apparatus 100 (e.g., within the apparatus 100 or on a computer display operatively coupled to the apparatus 100). In other embodiments of the invention, the output may be e-mailed, texted, instant messaged, or transferred through any other electronic means in order to provide the output to the desired user.

After the rinsing process in block 216, and after, before, or during the output verification process of block 216, the probe 110 is removed from the apparatus by disengaging the securing member 106 (e.g., holder, clamp, clip, or other fastener) and the probe 110 is dried according to the probe manufacturer's instructions, as illustrated by block 218 in FIG. 7. After drying, the apparatus 100 is ready for a new cycle immediately after the preceding cycle is completed. A single cycle (e.g., from insertion of a probe 110 to the removal of the probe 110 after cleaning and disinfecting) may take approximately 20 minutes. It should be understood that in other embodiments of the invention the cycle may be less than 20 minutes or greater than 20 minutes (e.g., may be 10, 12, 14, 16, 18, 20, 22, 24, 26, 28, 30, or the like minutes, or range between, overlap, or fall outside any of these values).

Referred to throughout the present disclosure, the control unit assembly 103 (e.g., such as to the user interface, computer output, or various computer memory devices therein), may include a processor, memory, input/output (I/O) device, and a storage device. The control unit assembly 103 may also include a high-speed interface connecting to the memory, and a low-speed interface connecting to a low speed bus and storage device. Each of the components may be operatively coupled to one another using various buses and may be mounted on a common motherboard or in other manners as appropriate. As described herein, the processor may include a number of subsystems to execute the portions of processes described herein. Each subsystem may be a self-contained component of a larger system (e.g., control unit assembly 103) and capable of being configured to execute specialized processes as part of the larger system.

The processor can process instructions, such as instructions of an application that may perform the functions disclosed herein. These instructions may be stored in the memory (e.g., non-transitory storage device) or on the storage device, for execution within the control unit assembly 103 using any subsystems described herein. It is to be understood that the control unit assembly 103 may use, as appropriate, multiple processors, along with multiple memories, and/or I/O devices, to execute the processes described herein.

The memory stores information within the control unit assembly 103. In one implementation, the memory is a volatile memory unit or units, such as volatile random-access memory (RAM) having a cache area for the temporary storage of information, such as a command, instructions related to various methods and/or functionalities described herein, and/or the like. In another implementation, the memory is a non-volatile memory unit or units. The memory may also be another form of computer-readable medium, such as a magnetic or optical disk, which may be embedded and/or may be removable. The non-volatile memory may additionally or alternatively include an EEPROM, flash memory, and/or the like for storage of information such as instructions and/or data that may be read during execution of computer instructions. The memory may store, recall, receive, transmit, and/or access various files and/or information used by the control unit assembly 103 during operation.

The storage device is capable of providing mass storage for the control unit assembly 103. In one aspect, the storage device may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier may be a non-transitory computer-or machine-readable storage medium, such as the memory, the storage device, or memory on processor.

The high-speed interface manages bandwidth-intensive operations for the control unit assembly 103, while the low speed controller manages lower bandwidth-intensive operations. Such allocation of functions is exemplary only. In some embodiments, the high-speed interface is coupled to memory, input/output (I/O) device (e.g., through a graphics processor or accelerator), and to high-speed expansion ports, which may accept various expansion cards (not shown). In such an implementation, low-speed controller is coupled to storage device and low-speed expansion port. The low-speed expansion port, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The processor may be configured to communicate with the user through a control interface and display interface coupled to a display. The display may be, for example, a TFT LCD (Thin-Film-Transistor Liquid Crystal Display) or an OLED (Organic Light Emitting Diode) display, or other appropriate display technology. The display interface may comprise appropriate circuitry and configured for driving the display to present graphical and other information to a user. The control interface may receive commands from a user and convert them for submission to the processor. In addition, an external interface may be provided in communication with processor, so as to enable near area communication of control unit assembly 103 with other devices. The external interface may provide, for example, for wired communication in some implementations, or for wireless communication in other implementations, and multiple interfaces may also be used.

Embodiments of the invention provide improvements over traditional systems in numerous ways. Traditional probe disinfecting apparatuses may implement a plurality of heaters to control the temperatures of various circuits (e.g., a first heater for the disinfecting liquid and a second heater for the rinsing liquid and/or cleaner liquid). In contrast, the apparatus described herein improves upon such traditional designs by incorporating a plurality of circuits into a single heater housing with one heating element. The dual heater system described herein supplies heat to the multiple circuits (e.g., disinfectant liquid, rinsing liquid, and/or cleaner liquid). As a result, manufacturing costs may be reduced by only requiring the fabrication of a single set of parts for a single heater instead of multiple sets of parts for a plurality of heaters. Similarly, operational and maintenance costs of the apparatus may be lower than traditional systems using a plurality of heaters, as the replacement of a single heater with fewer parts to disassemble and/or service results in shorter times for servicing and fewer replacement parts.

As it compares to other traditional probe cleaners without the use of any heaters, embodiments described herein implementing the dual heater are more efficient in disinfecting probes due to the elevated temperature of the disinfectant liquid, rinsing liquid, and/or cleaner liquid for application to a probe. The elevated temperature of such disinfectant liquid, rinsing liquid, and/or cleaner liquid provides an increase in energy to the liquid molecules to facilitate the breakdown of bonds between contaminants and the surfaces the contaminants are adhered to.

The nozzle system described herein improves upon traditional systems by strategically positioning and spacing one or more nozzles at distances and angles from the probe such that the spray patterns from each of the one or more nozzles overlap with that of an adjacent nozzle. In doing so, the coverage of the disinfectant liquid, rinsing liquid, and/or cleaner liquid is maximized while not being redundantly applied, which significantly reduces the amount of disinfectant liquid, rinsing liquid, and/or cleaner liquid required for disinfecting the probe(s). As a result, the operational costs of consumables (such as the disinfectant liquid, rinsing liquid, and/or cleaner liquid), time, and energy required for the apparatus to clean probe(s) are minimized, which also reduces the environmental impact of the apparatus by preventing the unnecessary waste of disinfectant liquid, rinsing liquid, and/or cleaner liquid.

Moreover, traditional systems with lower-pressure or zero-pressure application of such disinfectant liquid, rinsing liquid, and/or cleaner liquid may require additional mechanical scrubbing of the probe(s) and/or longer operational times such that the probe(s) may stay in contact with the disinfectant liquid, rinsing liquid, and/or cleaner liquid for an extended time period. The use of nozzles as described in the present disclosure forcibly removes contaminants by using high-pressure application of disinfectant liquid, rinsing liquid, and/or cleaner liquid to the surface of the probe(s). By forcibly removing contaminants using high-pressure application, the disinfecting times, amount of disinfectant liquid, rinsing liquid, and/or cleaner liquid required, and energy usage are reduced.

The positioning system described herein provides improvements to traditional systems by facilitating the proper positioning of the probe(s) relative to the nozzle(s). A misaligned or improperly positioned probe may lead to portions of the probe(s) not receiving the desired volume and/or coverage of disinfectant liquid, rinsing liquid, and/or cleaner liquid. Such an event may require additional reprocessing of the probe(s), lengthening the processing time, increasing the usage of disinfectant liquid, rinsing liquid, and/or cleaner liquid, and requiring an increased level of operator interaction. By ensuring the proper positioning of the probe(s) using the positioning system described herein, the nozzle(s) to disinfect the probe(s) are implemented in an efficient manner such that coverage of disinfectant liquid, rinsing liquid, and/or cleaner liquid on the probe(s) is maximized. In doing so, the amount of time required for disinfecting the probe(s) is minimized and the amount of disinfectant liquid, rinsing liquid, and/or cleaner liquid required is also similarly minimized.

Unless otherwise defined, all terms (including technical and scientific terms) used herein have the same meaning as commonly understood by one of ordinary skill in the art to which this invention belongs. It will be further understood that terms used herein should be interpreted as having a meaning that is consistent with their meaning in the context of this specification and the relevant art and should not be interpreted in an idealized or overly formal sense unless expressly so defined herein. Certain terminology is used herein for convenience only and is not to be taken as a limitation on the invention. For example, words such as “upper,” “lower,” “left,” “right,” “horizontal,” “vertical,” “upward,” and “downward” merely describe the configuration shown in the figures. The referenced components may be oriented in an orientation other than that shown in the drawings and the terminology, therefore, should be understood as encompassing such variations unless specified otherwise.

It will be understood that when an element is referred to as being “connected,” “coupled,” or “operatively coupled” to another element, the elements can be formed integrally with each other, or may be formed separately and put together. Furthermore, “connected,” “coupled,” or “operatively coupled” to can mean the element is directly connected, coupled, or operatively coupled to the other element, or intervening elements may be present between the elements. Furthermore, “connected,” “coupled,” or operatively coupled” may mean that the elements are detachable from each other, or that they are permanently coupled together.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations, modifications, and combinations of the just described embodiments can be configured without departing from the scope and spirit of the invention. Therefore, it is to be understood that, within the scope of the appended claims, the invention may be practiced other than as specifically described herein.

What is claimed is:

1. An apparatus for disinfecting a probe, the apparatus comprising:
a probe reservoir assembly, wherein the probe reservoir assembly is configured for securing the probe and dispensing a liquid onto the probe, wherein the liquid is a disinfectant liquid or a rinsing liquid;
a disinfectant assembly operatively coupled to the probe reservoir assembly, wherein the disinfectant assembly is configured to provide the disinfectant liquid for the disinfecting of the probe; and
a rinsing assembly operatively coupled to the probe reservoir assembly, wherein the rinsing assembly is configured to provide the rinsing liquid for rinsing the probe.

2. The apparatus of claim 1, wherein the probe reservoir assembly comprises:
a probe reservoir;
a securing member for securing the probe within the reservoir; and
a one or more nozzles that dispense the liquid on the probe within the probe reservoir, wherein the liquid is the disinfectant liquid for the disinfecting or the rinsing liquid for the rinsing, and wherein the one or more nozzles dispense the liquid in a conical pattern.

3. The apparatus of claim 2, wherein the one or more nozzles comprise a plurality of nozzles, wherein the plurality of nozzles provide overlapping coverage of the liquid on the probe, and wherein the liquid runs-off the probe by gravity.

4. The apparatus of claim 1, further comprising:
a heater assembly comprising:
a heater comprising a heating element;
a first circuit operatively coupled to the disinfectant assembly; and
a second circuit operatively coupled to the rinsing assembly.

5. The apparatus of claim 4, wherein the heater comprises:
a first channel for the first circuit, wherein the first channel receives less than half of the heat from the heating element; and
a second channel for the second circuit, wherein the second channel receives greater than half of the heat from the heating element.

6. The apparatus of claim 4, wherein the heater assembly further comprises:
a first temperature sensor for the first circuit, and wherein a disinfectant temperature of the heated disinfectant is controlled by electronically adjusting the heater element based on the first temperature sensor.

7. The apparatus of claim 4, wherein the heater assembly further comprises:
a second temperature sensor for the second circuit, and wherein a rinsing temperature of the rinsing liquid is controlled by adjusting water suppled to the rinsing liquid for quenching based on the second temperature sensor.

8. The apparatus of claim 4, wherein the first circuit has a controlled volume and the second circuit has a variable volume.

9. The apparatus of claim 4, wherein the disinfectant assembly further comprises:
a mixing member within the first circuit, wherein the mixing member is configured to mix the heated disinfectant liquid to increase the uniformity of the heated disinfectant liquid.

10. The apparatus of claim 8, wherein the mixing member comprises:
a tortuous path mixing member that agitates the heated disinfectant liquid.

11. The apparatus of claim 1, further comprising:
a probe positioning assembly comprising:
a securing member for securing the probe;
one or more sensors; and
a processor in electrical communication with the one or more sensors configured to receive a signal from the one or more sensors;
wherein the one or more sensors are configured to capture indicator information from an indicator, wherein the indicator is operatively coupled to the probe; or
wherein the one or more sensors are configured to capture presence information from the probe.

12. The apparatus of claim 11, wherein the indicator is operatively coupled to the cord of the probe.

13. The apparatus of claim 11, wherein the one or more sensors comprise a reflective object sensor and the indicator is a color, a hall-effect sensor and the indicator is a magnet, or a wireless identification reader and the indicator is a tag.

14. The apparatus of claim 11, wherein the one or more sensors is one or more indicator sensors comprising a first indicator sensor and the indicator information is captured from a first portion of the indicator, and wherein the signal is a first signal corresponding to the first portion of the indicator.

15. The apparatus of claim 14, wherein the one or more indicator sensors further comprises a second indicator sensor and the indicator information is further captured from a second portion of the indicator, and wherein processor receives a second signal corresponding to the second portion of the indicator, and wherein the processor determines a position of the probe based on the indicator information captured from the first portion and second portion of the indicator.

16. The apparatus of claim 15, wherein the first sensor and the second sensor are reflective object sensors, wherein the first portion of the indicator is a non-reflective portion and the second portion of the indicator is a reflective portion, and wherein the processor is configured to determine that the probe is in a correct position as a result of a condition where the first signal indicates the reflective portion is detected by the first sensor and the second signal indicates the non-reflective portion is detected by the second sensor.

17. The apparatus of claim 11, wherein the one or more sensors is the presence sensor, and wherein the signal is an indication of the presence of the probe.

18. The apparatus of claim 17, wherein the presence sensor is a momentary push button switch.

19. The apparatus of claim 11, wherein the securing member comprises:
an apparatus housing;
one or more rollers; and
one or more biasing members operatively coupled to the one or more rollers;
wherein a cord of the probe is configured to be held by the one or more rollers and the housing through the force provided by the one or more biasing members.

20. A method of disinfecting a probe, the method comprising:
disinfecting the probe via at least one nozzle, the at least one nozzle applying a disinfectant heated by a heater assembly in a first circuit; and
rinsing the probe with a rinse liquid heated by the heater assembly in a second circuit;
wherein the first circuit and second circuit share a heating element from the heater assembly.
